(19) United States
(12) Patent Application Publication
(10) Pub. No.: US 2022/0378727 A1
(43) Pub. Date: Dec. 1, 2022

(54) DRY POWDER FORMULATIONS OF TAMIBAROTENE FOR PULMONARY AND INTRANASAL DELIVERY

(71) Applicants: The University of Hong Kong, Hong Kong (CN); Centre for Virology, Vaccinology and Therapeutics Limited, Hong Kong (CN)

(72) Inventors: Ka Wing Lam, Hong Kong (CN); Shuofeng Yuan, Hong Kong (CN); Qiuying Liao, Hong Kong (CN); Kwok Yung Yuen, Hong Kong (CN)

(21) Appl. No.: 17/743,271

(22) Filed: May 12, 2022

Related U.S. Application Data
(60) Provisional application No. 63/188,793, filed on May 14, 2021.

(51) Int. Cl. A61K 31/167; A61K 9/00; A61K 9/19; A61K 47/40; A61K 47/55

(52) U.S. Cl. CPC A61K 31/167; A61K 9/0075; A61K 9/19; A61K 9/008; A61K 47/40; A61K 47/551

(57) ABSTRACT

Dry powder formulations for pulmonary and/or intranasal drug delivery, delivery systems, and methods of making and using thereof have been developed. The dry powder formulation contains particles containing a retinoid and/or a retinoid derivative, such as tamibarotene; and a β-cyclodextrin and/or a β-cyclodextrin derivative. The dry powder formulation allows for improved drug adsorption and bioavailability in vivo. The particles of the dry powder formulation have favorable aerodynamic properties for deposition and retention in the lower and/or upper respiratory tract(s). The dry powder formulation can be prepared using spray-drying or spray-freeze-drying. Inhalation, intratracheal administration, and/or intranasal administration of the dry powder formulation can deliver to a subject an effective amount of the retinoid and/or retinoid derivative to prevent, treat, or ameliorate symptom(s) associated with a respiratory viral infection in the subject.

### CROSS-REFERENCE TO RELATED APPLICATION

This application claims the benefit of and priority to U.S. Provisional Application No. 63/188,793, filed on May 14, 2021, which is hereby incorporated herein by reference in its entirety.

### FIELD OF THE INVENTION

This invention is generally directed to powder formulations for delivery of antiviral agents and methods for making and using thereof.

### BACKGROUND OF THE INVENTION

Respiratory viral infections are posing significant threat to global public health and heavy economic burden. Rapid and effective control of these epidemics at their onset are challenging due to the long-time lag to develop specific antiviral agents or vaccines. Early administration of a broad-spectrum antiviral agent can be an effective strategy to facilitate the control of an epidemic early on or even before the pathogen is identified. However, most of the currently available antiviral agents are administered by oral or intravenous administration, which is difficult to achieve robust antiviral activity in the respiratory tract due to inadequate lung distribution following oral or intravenous administration. When high dose is administered to compensate the insufficient lung distribution, toxic side effects are expected because of the extensive systemic exposure.

There remains a need for formulations of broad-spectrum antiviral agents for pulmonary or intranasal delivery.

Therefore, it is the object of the present invention to provide dry powder formulations for delivery of antiviral agents by inhalation or intranasal administration.

It is another object of the present invention to provide methods of making the dry powder formulations.

It is yet another object of the present invention to provide methods of using the dry powder formulations.

### SUMMARY OF THE INVENTION

Dry powder formulations for pulmonary and intranasal drug delivery, delivery systems for the dry powder formulations, and methods of making and using thereof have been developed.

The dry powder formulation contains particles containing (1) a retinoid or retinoid derivative, or a combination thereof, such as tamibarotene; and (2) a β-cyclodextrin or a β-cyclodextrin derivative, or a combination thereof, where the amount of the β-cyclodextrin or the β-cyclodextrin derivative, or the total amount of the β-cyclodextrin and β-cyclodextrin derivative is at least 20 wt % of the total amount of the retinoid and/or retinoid derivative and the β-cyclodextrin and/or β-cyclodextrin derivative. The particles are typically porous and spherical in shape. The retinoid and/or retinoid derivative can form a complex with the β-cyclodextrin and/or β-cyclodextrin derivative via hydrophobic interactions. The retinoid and/or retinoid derivative in the complex are/is in an amorphous form.

The particles of the dry powder formulation have favorable aerodynamic properties for effective respiratory tract deposition and retention. For example, the particles of the dry powder formulation have a mass median aerodynamic diameter (“MMAD”)<5 μm, <4 μm, <3.5 μm, <3 μm, <2.5 μm, or <2 μm; a volumetric mean diameter that is larger than the MMAD, such as >4 μm, >5 μm, >8 μm, >10 μm, >12 μm, >15 μm, in a range from 4 μm to 20 μm, from 4 μm to 15 μm, or from 4 μm to 15 μm; and/or a fine particle fraction>40%, >45%, >50%, >55%, >60%, or ≥65% in cascade impactor study, allowing for effective lung deposition and retention. For example, the particles of the dry powder formulation have a MMAD>9 μm, >9.5 μm, >10 μm, or >10.5 μm; a volumetric mean diameter>50 μm, >55 μm, >60 μm, >65 μm, in a range from 50 μm to 80 μm, from 50 μm to 75 μm, or from 50 μm to 70 μm; and/or a fraction of particles>9 μm of >40%, >45%, >50%, >55%, or >60% in Andersen Cascade Impactor (“ACI”) study, allowing for effective deposition and retention in the upper respiratory tract.

In some aspects, the dry powder formulation contains particles containing a retinoid derivative, such as tretinoin, isotretinoin, alitretinoin, etretinate, acitretin, adapalene, bexarotene, tazarotene, or tamibarotene and a β-cyclodextrin derivative, and the β-cyclodextrin derivative can have a degree of substitution of from 1 to 21, from 1 to 12, from 2 to 15, from 2 to 12, from 2 to 10, from 4 to 10, from 5 to 8, from 6 to 9, or from 6 to 8. In some aspects, the retinoid derivative contained in the particles of the dry powder formulation can be tamibarotene. In some aspects, the β-cyclodextrin derivative contained in the particles of the dry powder formulation can be 2-hydroxypropyl-β-cyclodextrin, methyl-β-cyclodextrin, or sulfobutylether β-cyclodextrin, or a combination thereof. In some aspect, the only β-cyclodextrin derivative contained in the particles of the dry powder formulation is 2-hydroxypropyl-β-cyclodextrin.

The dry powder formulation may further contain a pharmaceutically acceptable excipient or an additional active agent, or a combination thereof. The pharmaceutically acceptable excipient can be an amino acid, a peptide, a lipid, a protein, a chelating agent, a salt, a taste masking agent, a cation, or a polymer, or a combination thereof. The amount of the pharmaceutically acceptable excipient in the dry powder formulation can be in a range from 0.1 wt % to 20 wt %, from, from 0.1 wt % to 15 wt %, or from 1 wt % to 10 wt % of the dry powder formulation. The additional active agent can be an anti-inflammatory agent or an anti-viral agent, or a combination thereof. In some aspects, the dry powder formulation does not contain any pharmaceutically acceptable excipients.

Delivery systems containing an inhaler or a nasal device and the dry powder formulation are provided. In some aspects, the dry powder formulation is formulated into a solution or a suspension using a suitable solvent for intranasal administration as drops or a spray. The emitted fraction of particles using the disclosed delivery system can be >65%, >70%, >75%, >80%, >85%, >90%, >92%, or >95%.

Also provided are methods of making the dry powder formulation. Generally, the method includes (i) mixing a retinoid and/or a retinoid derivative and a β-cyclodextrin and/or a β-cyclodextrin derivative, and optionally a pharmaceutically acceptable excipient and/or an additional active agent, in a solvent to form a liquid feed; and (ii) spray-drying or spray-freeze drying the liquid feed to form particles containing the retinoid and/or retinoid derivative and the β-cyclodextrin and/or β-cyclodextrin derivative, and optionally the pharmaceutically acceptable excipient and/or the additional active agent. Typically, following step (ii), the production yield of the particles is at least 40 wt %, such as from about 40 wt % to about 95 wt % or from about 65 wt % to about 95 wt %.

Further provided are methods of preventing, treating, or ameliorating symptom(s) associated with a respiratory viral infection in a subject. The subject being treated can be a mammal having or at the risk of having a respiratory viral infection, such as sever acute respiratory syndrome, Middle East respiratory syndrome, Coronavirus Disease, or a flu caused by an influenza virus, or a combination thereof. Generally, the method includes (i) administering to the subject the dry powder formulation. The administration step may be repeated one or more times. For example, the administration step is repeated every hour, every 2 hours, every 5 hours, every 8 hours, every day, every 2 days, every 3 days, every 5 days, every 7 days, every 10 days, every two weeks, or every month. The period for repeated administration of the dry powder formulation can be between one day and 6 months, between one day and 3 months, between one and thirty days, between one and ten days, between one and three days, between one and two days, or during one day.

Typically, following a single administration step or all of the administration steps, an effective amount of the retinoid and/or retinoid derivative is delivered to the lower and/or upper respiratory tract of the subject.

### BRIEF DESCRIPTION OF THE DRAWINGS

FIG. 1A is a schematic showing the complex formed by a hydrophobic drug and cyclodextrin. FIGS. 1B and 1C are schematics of the two steps involved in spray freeze drying: spray freezing, atomization of liquid by a nozzle into cryogen forming frozen particles (FIG. 1B) and freeze drying, sublimation of solvent and formation of dried porous particles (FIG. 1C). FIG. 1D is a schematic illustrating spherical porous dry powder produced by spray freeze drying. FIG. 1E is a scanning electron microscopy (“SEM”) image of spherical porous dry powder produced by spray freeze drying, under 5000× magnification. FIG. 1F is a schematic illustrating oral inhalation of porous particles to deliver drugs into the deep lung region.

FIGS. 2A-2H are graphs showing the physicochemical and aerosol properties of spray freeze dried tamibarotene powder (A2-TFN). FIGS. 2A-2D are SEM images of unformulated tamibarotene at 1.0 k (FIG. 2A) and 5.0 k (FIG. 2C), and A2-TFN at 1.0 k (FIG. 2B) and 5.0 k (FIG. 2D). FIG. 2E is a bar graph showing the aerosol performance of A2-TFN powders evaluated by Next Generation Impactor (NGI) operated at 90 L/min, with the use of Breezhaler® for powder dispersion. EF: emitted fraction (fraction of particle that exited the inhaler); FPF: fine particle fraction (fraction of particle with aerodynamic diameter below 5 μm). FIG. 2F is a graph showing the dissolution profile of fine particle dose (FPD—mass of particles with aerodynamic diameter below 5 μm) of A2-TFN compared with unformulated tamibarotene. Simulated lung fluid was used as dissolution medium. Data were presented as mean±standard deviation (n=3). FIGS. 2G and 2H are graphs showing the Fourier-transform infrared spectroscopy (“FT-IR”) spectra (FIG. 2G) and differential scanning calorimetry (“DSC”) thermograms of unformulated tamibarotene, HPBCD, physical mixture of HPBCD and tamibarotene, and A2-TFN powder (FIG. 2H). Negative peak in DSC thermogram represents endothermic event. The terms “HPBCD” and “HPβCD” are used interchangeably herein.

FIGS. 3A and 3B are SEM images of A2-US powder produced by spray freeze drying coupled with an ultrasonic nozzle at 1.0 k (FIG. 3A) and 5.0 k (FIG. 3B). The large particles were designed for intranasal delivery. b, A1-SD powder produced by spray drying. The size particles were designed for oral inhalation delivery. FIGS. 3C and 3D are SEM images of A1-SD powder produced by spray drying at 5.0 k (FIG. 3C) and 10.0 k (FIG. 3D).

FIGS. 4A and 4B are bar graphs showing in vivo biodistribution of A2-TFN-fluorescein powder in the lung (FIG. 4A) and kidney (FIG. 4B) at 0.5 h and 1 hour after intratracheal administration. The fluorescence images were acquired with an IVIS Spectrum in vivo imaging system with excitation and emission wavelengths of 465 and 540 nm, respectively. Fluorescence intensities of lung and kidney regions were quantified using the IVIS Spectrum imaging system. Data is presented as mean±standard deviation (n=2-4).

FIGS. 5A and 5B are schematics illustrating the administration of tamibarotene to healthy BALB/c mice as a A2-TFN powder formulation via the intratracheal (i.t.) route (FIG. 5A) or as an unformulated suspension (in 0.1% DMSO) via the intraperitoneal (i.p.) route (FIG. 5B). A dose of 5 mg/kg tamibarotene was administered. FIG. 5C is a graph showing the tamibarotene concentration versus time in plasma. FIG. 5D is a graph showing the tamibarotene concentration versus time in the lung tissues. FIG. 5E is a graph showing a zoom-in view of the i.p. curve shown in FIG. 5D. Data were presented as mean±standard deviation (n=5).

FIG. 6A is a schematic illustrating the experimental protocol in Syrian hamster for testing the in vivo antiviral activity of tamibarotene formulations against SARS-CoV-2 for prophylactic protection. Each hamster received either PBS solution, A2-TFN powder or remdesivir solution via intratracheal (i.t.) administration prior to intranasal (i.n.) inoculation of SARS-CoV-2. FIG. 6B is a graph showing the viral loads in the lung tissue of the infected hamsters. Lung tissue of the infected hamsters were harvested at 4 days post-infection for viral load assay (n=3 for remdesivir group and n=4 for PBS and A2-TFN groups). Differences were compared by one-way ANOVA with post-hoc multiple comparison. **p<0.01, compared with PBS group. FIG. 6C is a graph showing the SARS-CoV-2 titers in the lung tissue of the infected hamsters. Lung tissues of the infected hamsters were harvested for plaque assay. Detection limit: 100 p.f.u./ml. Two samples in A2-TFN group were below the detection limit. For statistical purpose, a value of 100 is used for these two samples.

FIG. 7A is a schematic illustrating the experimental protocol in human dipeptidyl peptidase (hDPP4) transgenic C57BL/6 mice for testing the in vivo antiviral activity of tamibarotene formulations against MERS-CoV for prophylactic protection. Each mouse received either PBS, A2-TFN powder or unformulated tamibarotene suspension via intratracheal (i.t.) administration prior to intranasal (i.n.) inoculation of MERS-CoV. FIG. 7B is a graph showing the viral loads in the lung tissues of the infected mice. Lung tissues were harvested at 3 days post-infection for viral RNA load assay (n=5). Differences were compared by one-way ANOVA with post-hoc multiple comparison. ** p<0.01, ***p<0.001, compared with PBS group.

FIG. 8A is a schematic illustrating the experimental protocol in BALB/c mice for testing the in vivo antiviral activity of tamibarotene formulations against H1N1 virus for prophylactic protection. Each mouse received either PBS, unformulated tamibarotene suspension, A2-TFN powder or zanamivir solution via i.t. administration prior to intranasal (i.n.) inoculation of H1N1 virus. FIG. 8B is a graph showing the survival rates of the infected mice. Survivals were monitored for 14 days or until the human endpoint was reached (n=5). The survival rates in each group were compared using Log-rank test (Mantel-Cox). **p<0.01, compared with PBS group. FIG. 8C is a graph showing the body weights of the infected mice. Daily body weights of surviving mice were presented as percentage of baseline level (n=5). FIG. 8D is a graph showing the body weight of mice in a toxicity study of SFD tamibarotene powder formulation (A2-TFN) and HPBCD. FIG. 8E is a graph showing the viral loads in the lung tissues of the infected mice. Lung tissues were harvested at 3 days post-infection for viral load assay (n=4). Differences were compared by one-way ANOVA with post-hoc multiple comparison. **p<0.01, ns not significant, compared with PBS group.

FIG. 9A is a schematic illustrating the experimental protocol in BALB/c mice for testing the in vivo antiviral activity of tamibarotene formulations against H1N1 virus for therapeutic intranasal (i.n.) treatment. FIG. 9B is a graph showing the survival rates of the infected mice. Survivals were monitored for 14 days or until the human endpoint was reached (n=7). The survival rates in each group were compared using Log-rank test (Mantel-Cox). ***p<0.001, **p<0.01, compared with PBS group. FIG. 9C is a graph showing the body weight of the infected mice. Daily body weights of surviving mice were presented as percentage of baseline level (n=7). FIG. 9D is a graph showing the viral loads in the lung tissues of the infected mice. Lung tissues were harvested at 3 days post-infection for viral load assay (n=4). Differences were compared by one-way ANOVA with post-hoc multiple comparison. *p<0.05, ns not significant, compared with PBS group.

### DETAILED DESCRIPTION OF THE INVENTION

### I. Definitions

As used herein, the term “active agent” refers to a physiologically or pharmacologically active substance that acts locally and/or systemically in the body. An active agent is a substance that is administered to a patient for the treatment (e.g., therapeutic agent), prevention (e.g., prophylactic agent), or diagnosis (e.g., diagnostic agent) of a disease or disorder.

As used herein, the term “pharmaceutically acceptable” as used herein refers to those compounds, materials, and/or compositions, which are, within the scope of sound medical judgment, suitable for use in contact with the tissues of human beings and animals without excessive toxicity, irritation, allergic response, or other problems or complications commensurate with a reasonable benefit/risk ratio.

As used herein, the term “alkyl” refers to univalent groups derived from alkanes by removal of a hydrogen atom from any carbon atom. Alkanes represent saturated hydrocarbons, including those that are linear, branched, or cyclic (either monocyclic or polycyclic). An alkyl can be a linear C₁-C₃₀ alkyl, a branched C₄-C₃₀ alkyl, a cyclic C₃-C₃₀ alkyl, a linear C₁-C₃₀ alkyl or a branched C₄-C₃₀ alkyl, a linear C₁-C₃₀ alkyl or a cyclic C₃-C₃₀ alkyl, a branched C₄-C₃₀ alkyl or a cyclic C₃-C₃₀ alkyl. Optionally, alkyl groups have up to 20 carbon atoms. An alkyl can be a linear C₁-C₂₀ alkyl, a branched C₄-C₂₀ alkyl, a cyclic C₃-C₂₀ alkyl, a linear C₁-C₂₀ alkyl or a branched C₄-C₂₀ alkyl, a branched C₄-C₂₀ alkyl or a cyclic C₃-C₂₀ alkyl, a linear C₁-C₂₀ alkyl or a cyclic C₃-C₂₀ alkyl. Optionally, alkyl groups have up to 10 carbon atoms. An alkyl can be a linear C₁-C₁₀ alkyl, a branched C₄-C₁₀ alkyl, a cyclic C₃-C₁₀ alkyl, a linear C₁-C₁₀ alkyl or a branched C₄-C₁₀ alkyl, a branched C₄-C₁₀ alkyl or a cyclic C₃-C₁₀ alkyl, a linear C₁-C₁₀ alkyl or a cyclic C₃-C₁₀ alkyl. Optionally, alkyl groups have up to 6 carbon atoms. An alkyl can be a linear C₁-C₆ alkyl, a branched C₄-C₆ alkyl, a cyclic C₃-C₆ alkyl, a linear C₁-C₆ alkyl or a branched C₄-C₆ alkyl, a branched C₄-C₆ alkyl or a cyclic C₃-C₆ alkyl, or a linear C₁-C₆ alkyl or a cyclic C₃-C₆ alkyl. Optionally, alkyl groups have up to four carbons. An alkyl can be a linear C₁-C₄ alkyl, cyclic C₃-C₄ alkyl, a linear C₁-C₄ alkyl or a cyclic C₃-C₄ alkyl. Preferably, the alkyl group is unsubstituted alkyl group. Preferably, the alkyl group is a linear C₁-C₅, C₁-C₄, C₁-C₃, C₁-C₂ alkyl group, such as methyl group.

As used herein, the term “heteroalkyl” refers to alkyl groups where one or more carbon atoms are replaced with a heteroatom, such as, O, N, or S. Heteroalkyl group can be linear, branched, or cyclic. A heteroalkyl can be a linear C₁-C₃₀ heteroalkyl, a branched C₃-C₃₀ heteroalkyl, a cyclic C₂-C₃₀ heteroalkyl, a linear C₁-C₃₀ heteroalkyl or a branched C₃-C₃₀ heteroalkyl, a linear C₁-C₃₀ heteroalkyl or a cyclic C₂-C₃₀ heteroalkyl, a branched C₃-C₃₀ heteroalkyl or a cyclic C₂-C₃₀ heteroalkyl. Optionally, heteroalkyl groups have up to 20 carbon atoms. A heteroalkyl can be a linear C₁-C₂₀ heteroalkyl, a branched C₃-C₂₀ heteroalkyl, a cyclic C₂-C₂₀ heteroalkyl, a linear C₁-C₂₀ heteroalkyl or a branched C₃-C₂₀ heteroalkyl, a branched C₃-C₂₀ heteroalkyl or a cyclic C₂-C₂₀ heteroalkyl, or a linear C₁-C₂₀ heteroalkyl or a cyclic C₂-C₂₀ heteroalkyl. Optionally, heteroalkyl groups have up to 10 carbon atoms. A heteroalkyl can be a linear C₁-C₁₀ heteroalkyl, a branched C₃-C₁₀ heteroalkyl, a cyclic C₂-C₁₀ heteroalkyl, a linear C₁-C₁₀ heteroalkyl or a branched C₃-C₁₀ heteroalkyl, a branched C₃-C₁₀ heteroalkyl or a cyclic C₂-C₁₀ heteroalkyl, or a linear C₁-C₁₀ heteroalkyl or a cyclic C₂-C₁₀ heteroalkyl. Optionally, heteroalkyl groups have up to 6 carbon atoms. A heteroalkyl can be a linear C₁-C₆ heteroalkyl, a branched C₃-C₆ heteroalkyl, a cyclic C₂-C₆ heteroalkyl, a linear C₁-C₆ heteroalkyl or a branched C₃-C₆ heteroalkyl, a branched C₃-C₆ heteroalkyl or a cyclic C₂-C₆ heteroalkyl, or a linear C₁-C₆ heteroalkyl or a cyclic C₂-C₆ heteroalkyl. Optionally, heteroalkyl groups have up to four carbons. A heteroalkyl can be a linear C₁-C₄ heteroalkyl, a branched C₃-C₄ heteroalkyl, a cyclic C₂-C₄ heteroalkyl, a linear C₁-C₄ heteroalkyl or a branched C₃-C₄ heteroalkyl, a branched C₃-C₄ heteroalkyl or a cyclic C₂-C₄ heteroalkyl, or a linear C₁-C₄ heteroalkyl or a cyclic C₂-C₄ heteroalkyl.

As used herein, the term “alkenyl” refers to univalent groups derived from alkenes by removal of a hydrogen atom from any carbon atom. Alkenes are unsaturated hydrocarbons that contain at least one carbon-carbon double bond. Alkenyl group can be linear, branched, or cyclic. An alkenyl can be a linear C₂-C₃₀ alkenyl, a branched C₄-C₃₀ alkenyl, a cyclic C₃-C₃₀ alkenyl, a linear C₂-C₃₀ alkenyl or a branched C₄-C₃₀ alkenyl, a linear C₂-C₃₀ alkenyl or a cyclic C₃-C₃₀ alkenyl, a branched C₄-C₃₀ alkenyl or a cyclic C₃-C₃₀ alkenyl. Optionally, alkenyl groups have up to 20 carbon atoms. An alkenyl can be a linear C₂-C₂₀ alkenyl, a branched C₄-C₂₀ alkenyl, a cyclic C₃-C₂₀ alkenyl, a linear C₂-C₂₀ alkenyl or a branched C₄-C₂₀ alkenyl, a linear C₂-C₂₀ alkenyl or a cyclic C₃-C₂₀ alkenyl, a branched C₄-C₂₀ alkenyl or a cyclic C₃-C₂₀ alkenyl. Optionally, alkenyl groups have two to 10 carbon atoms. An alkenyl can be a linear C₂-C₁₀ alkenyl, a branched C₄-C₁₀ alkenyl, a cyclic C₃-C₁₀ alkenyl, a linear C₂-C₁₀ alkenyl or a branched C₄-C₁₀ alkenyl, a linear C₂-C₁₀ alkenyl or a cyclic C₃-C₁₀ alkenyl, a branched C₄-C₁₀ alkenyl or a cyclic C₃-C₁₀ alkenyl. Optionally, alkenyl groups have two to 6 carbon atoms. An alkenyl can be a linear C₂-C₆ alkenyl, a branched C₄-C₆ alkenyl, a cyclic C₃-C₆ alkenyl, a linear C₂-C₆ alkenyl or a branched C₄-C₆ alkenyl, a linear C₂-C₆ alkenyl or a cyclic C₃-C₆ alkenyl, a branched C₄-C₆ alkenyl or a cyclic C₃-C₆ alkenyl. Optionally, alkenyl groups have two to four carbons. An alkenyl can be a linear C₂-C₄ alkenyl, a cyclic C₃-C₄ alkenyl, a linear C₂-C₄ alkenyl or a cyclic C₃-C₄ alkenyl.

As used herein, the term “amino” includes the group NH₂ (primary amino), alkylamino (secondary amino), and dialkylamino (tertiary amino), where the two alkyl groups in dialkylamino may be the same or different, i.e., alkylalkylamino. Illustratively, amino include methylamino, ethylamino, dimethylamino, methylethylamino, and the like. In addition, it is to be understood that when amino modifies or is modified by another term, such as aminoalkyl, or acylamino, the above variations of the term amino continue to apply. Illustratively, aminoalkyl includes H²N-alkyl, methylaminoalkyl, ethylaminoalkyl, dimethylaminoalkyl, methylethylaminoalkyl, and the like. Illustratively, acylamino includes acylmethylamino, acylethylamino, and the like.

As used herein, the term “amide” includes the group CONH₂ (primary amide), CONHalkyl (secondary amide), and CONdialkyl (tertiary amide), where the two alkyl groups in CONdialkyl may be the same or different.

In a substituted functional group, one or more hydrogen atoms in the chemical group or moiety is replaced with one or more substituents. Any substitution is in accordance with permitted valence of the substituted atom and the substituent, and that the substitution results in a stable compound, i.e., a compound that does not spontaneously undergo transformation such as by rearrangement, cyclization, elimination, etc. Suitable substituents include, but are not limited to a halogen atom, an alkyl group, a cycloalkyl group, a heteroalkyl group, a cycloheteroalkyl group, an alkenyl group, a heteroalkenyl group, an alkynyl group, a heteroalkynyl group, an aryl group, a heteroaryl group, a polyaryl group, a polyheteroaryl group, —OH, —SH, —NH₂, —N₃, —OCN, —NCO, —ONO₂, —CN, —NC, —ONO, —CONH₂, —NO, —NO₂, —ONH₂, —SCN, —SNCS, —CF₃, —CH₂CF₃, —CH₂C₁, —CHCl₂, —CH₂NH₂, —NHCOH, —CHO, —COCl, —COF, —COBr, —COOH, —SO₃H, —CH₂SO₂CH₃, —PO₃H₂, —OPO₃H₂, —P(═O)(OR^{T1′})(OR^{T2′}) —OP(═O)(OR^{T1′})(OR^{T2′}), —BR^{T1′}(OR^{T2′}), —B(OR^{T1′})(OR^{T2′}), or -G′R^{T1′} in which -T′ is —O—, —S—, —NR^{T2′}—, —C(═O)—, —S(═O)—, —SO₂—, —C(═O)O—, —C(═O)NR^{T2′}—, —OC(═O)—, —NR^{T2′}C(═O)—, —OC(═O)O—, —OC(═O)NR^{T2′}—, —NR^{T2′}C(═O)O—, —NR^{T2′}C(═O)NR^{T3′}—, —C(═S)—, —C(═S)S—, —SC(═S)—, —SC(═S)S—, —C(═NR^{T2′})—, —C(═NR^{T2′})O—, —C(═NR^{T2′})NR^{T3′}—, —OC(═NR^{T2′}) —NR^{T2′}C(═NR^{T3′})—, —NR^{T2′}SO₂—, —C(═NR^{T2′})NR^{T3′}—, —OC(═NR^{T2′})—, —NR^{T2′}C(═NR^{T3′})—, —NR^{T2′}SO₂—, —NR^{T2′}SO₂NR^{T3′}—, —NR^{T2′}C(═S)—, —SC(═S)NR^{T2′}—, —NR^{T2′}C(═S)S—, —NR^{T2′}C(═S)NR^{T3′}—, —SC(═NR^{T2′})—, —C(═S)NR^{T2′}—, —OC(═S)NR^{T2′}—, —NR^{T2′}C(═S)O—, —SC(═O)NR^{T2′}—, —NR^{T2′}C(═S)—, —SC(═O)—, —SC(═O)S—, —C(═S)O—, —OC(═S)—, —OC(═S)O—, —SO₂NR^{T2′}—, —BR^{T2′}—, or —PR^{T2′}—; where each occurrence of R^{T1′}, R^{T2′}, and R^{T3′} is, independently, a hydrogen atom, a halogen atom, an alkyl group, a heteroalkyl group, an alkenyl group, a heteroalkenyl group, an alkynyl group, a heteroalkynyl group, an aryl group, or a heteroaryl group.

Recitation of ranges of values herein are merely intended to serve as a shorthand method of referring individually to each separate value falling within the range, unless otherwise indicated herein, and each separate value is incorporated into the specification as if it were individually recited herein.

Use of the term “about” is intended to describe values either above or below the stated value in a range of approx. +/−10%.

### II. Dry Powder Formulations

Disclosed herein are dry powder formulations of retinoid and/or retinoid derivatives for inhalation or intratracheal administration, and/or for intranasal administration into the lower and/or upper airways of a subject. The dry powder formulation of retinoid and/or retinoid derivatives can be used as a pulmonary or intranasal deliverable broad-spectrum antiviral therapy against various respiratory viral infections. The respiratory tract is the portal of entry of viruses that cause respiratory infections, it is desirable to deliver the antiviral agents directly at the primary site of infections. The dry powder formulation for pulmonary and/or intranasal delivery can improve drug distribution in the lower and/or upper airways, such as the lung, while minimize systemic exposure, such that effective antiviral activity against different categories of viruses can be achieved without a high dose of drugs.

The dry powder formulation contains particles containing a retinoid or a retinoid derivative, or a combination thereof, and a β-cyclodextrin or a β-cyclodextrin derivative, or a combination thereof. The particles are typically porous and/or spherical in shape. For example, the particles are porous and spherical in shape.

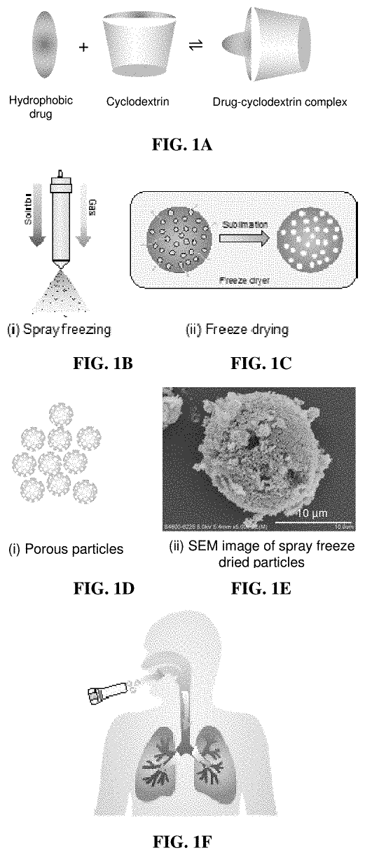

The retinoid and/or retinoid derivative are hydrophobic. Typically, the amount of the β-cyclodextrin or β-cyclodextrin derivative, or the total amount of the β-cyclodextrin and β-cyclodextrin derivative in the dry powder formulation is effective to enhance the solubility and the dissolution rate of the hydrophobic retinoid and/or retinoid derivatives. Without being bound to any theories, the β-cyclodextrin and/or β-cyclodextrin derivatives can enhance the solubility of the hydrophobic retinoid and/or retinoid derivatives by forming an inclusion complex. The β-cyclodextrin and/or β-cyclodextrin derivative contains a hydrophobic internal cavity and hydrophilic external surface. The retinoid and/or retinoid derivative can inert into the hydrophobic cavity of the β-cyclodextrin and/or β-cyclodextrin derivative and thus form a complex with the β-cyclodextrin and/or β-cyclodextrin derivative via hydrophobic interactions, thereby enhance the solubility of the hydrophobic retinoid and/or retinoid derivative. Additionally, the retinoid and/or retinoid derivative in the complex can be in an amorphous form instead of a crystalline form of an unformulated retinoid and/or retinoid derivative. The amorphous form of the retinoid and/or retinoid derivative can contribute to a faster dissolution rate of the drug. An exemplary schematic illustrating a complex formed by a hydrophobic retinoid or a hydrophobic retinoid derivative and a β-cyclodextrin or a β-cyclodextrin derivative via hydrophobic interactions is shown in FIG. 1A.

Generally, the amount of the β-cyclodextrin or β-cyclodextrin derivative, or the total amount of the β-cyclodextrin and β-cyclodextrin derivative is at least 20 wt % of the total amount of retinoid and/or retinoid derivative and the β-cyclodextrin and/or β-cyclodextrin derivative in the dry powder formulation. Generally, between 20 to 80 wt % of cyclodextrin can be used. An amount of cyclodextrin below 50 wt % is preferred, with lower amounts of cyclodextrin down to 20 wt % generally being more desirable. Generally, the amount can be adjusted depending on the molecular weight, aqueous solubility, and potency of the drug being used. Other types of cyclodextrin, such as α-cyclodextrin and γ-cyclodextrin and their derivatives, can be used so long as they are compatible with the drug being used.

For example, the weight ratio of the β-cyclodextrin and/or β-cyclodextrin derivative to the retinoid and/or retinoid derivative is in a range from 1:4 to 9:1, from 1:4 to 8.5:1, from 1:4 to 8:1, from 1:4 to 7.5:1, from 1:4 to 7:1, from 1:4 to 6.5:1, from 1:4 to 6:1, from 1:4 to 5.5:1, from 1:4 to 5:1, from 1:4 to 4.5:1, from 1:4 to 4:1, from 1:4 to 3.5:1, from 1:4 to 3:1, from 1:4 to 2.5:1, from 1:4 to 2:1, from 1:4 to 1.5:1, from 1:4 to 1:1, from 1:3 to 9:1, from 1:3 to 8.5:1, from 1:3 to 8:1, from 1:3 to 7.5:1, from 1:3 to 7:1, from 1:3 to 6.5:1, from 1:3 to 6:1, from 1:3 to 5.5:1, from 1:3 to 5:1, from 1:3 to 4.5:1, from 1:3 to 4:1, from 1:3 to 3.5:1, from 1:3 to 3:1, from 1:3 to 2.5:1, from 1:3 to 2:1, from 1:3 to 1.5:1, from 1:3 to 1:1, from 2:3 to 9:1, from 2:3 to 8.5:1, from 2:3 to 8:1, from 2:3 to 7.5:1, from 2:3 to 7:1, from 2:3 to 6.5:1, from 2:3 to 6:1, from 2:3 to 5.5:1, from 2:3 to 5:1, from 2:3 to 4.5:1, from 2:3 to 4:1, from 2:3 to 3.5:1, from 2:3 to 3:1, from 2:3 to 2.5:1, from 2:3 to 2:1, from 2:3 to 1.5:1, or from 2:3 to 1:1.

In some aspects, the particles of the dry powder formulation have favorable aerodynamic properties (e.g., mass median aerodynamic diameter (“MMAD”)<5 μm, volumetric mean diameter>4 μm and larger than the MMAD, and fine particle fraction>40% in cascade impactor study) for effective lung deposition and retention and improved drug dissolution rate, which lead to higher bioavailability in both the lung and plasma of a subject administered with the dry powder formulation and faster drug absorption in the subject.

For example, following inhalation or intratracheal administration of the dry powder formulation to a mammal, the maximum concentration of the retinoid and/or retinoid derivative delivered to the lung of the subject is at least 10-time higher, at least 15-time higher, at least 20-time higher, at least 25-time higher, at least 30-time higher, or at least 35-time higher than the maximum concentration of retinoid and/or retinoid derivative delivered to the lung of a control. Additionally or alternatively, the time to reach the maximum concentration of the retinoid and/or retinoid derivative in the lung of the subject is at least 2-time shorter, at least 3-time shorter, at least 4-time shorter, at least 5-time shorter, or at least 6-time shorter than the time to reach the maximum concentration of retinoid and/or retinoid derivative in the control. The control is the same species as the subject, which is administered with the same amount of retinoid and/or retinoid derivative as that in the dry powder formulation, but in an unformulated form, by intraperitoneal administration.

In some aspects, the particles of the dry powder formulation have aerodynamic properties (e.g., MMAD>9 μm, volumetric mean diameter>50 μm and larger than the MMAD, and fraction of particles>9 μm>40% in ACI study for intranasal administration, and improved drug dissolution rate. In general, MMAD above 10 μm is effective for deposition and retention in the nasal cavity upon aerosolization. In ACI equipped with glass expansion chamber (for nasal deposition) typically has an MMAD cut-off at 9 μm, hence particles with MMAD>9 μm or MMAD>10 μm are generally accepted to be suitable for intranasal administration. Generally, there is low direct correlation between volumetric diameter and the suitability for nasal administration. However, in general, larger the volumetric diameter, the larger the MMAD, with MMAD also depending on the shape and density of the particles. Thus, a selected MMAD will generally set the range of volumetric diameter of the particles.

The improved dissolution rate of the dry powder formulation for inhalation, intratracheal and intranasal administration can be attributed to the effect of porous structure of the particles and the complexation between the hydrophobic drug (i.e., the retinoid and/or retinoid derivatives) and the β-cyclodextrin and/or β-cyclodextrin derivatives. Additionally, particles with small aerodynamic diameter but large volumetric size can contribute to efficient lung deposition yet prolonged retention in the airway by avoiding rapid clearance.

The dry powder formulation may also contain a pharmaceutically acceptable carrier and/or an additional active agent.

Systems for delivering the dry powder formulations are also disclosed. In some aspects, the system includes an inhaler and the dry powder formulation. The emitted fraction of the particles of the powder formulation, i.e., the fraction of powders exited the inhaler, is >65%. In some aspects, the system includes a nasal device and a solution or suspension formed by the dry powder formulation and a suitable solvent. The emitted fraction of the particles of the solution or suspension, i.e., the fraction of powders exited the nasal device, is >85%. The emitted fraction can be measured by, for example, quantify the drug content using HPLC method after collecting the dissolved powder (in a solution form). Emitted fraction is calculated as the percentage recovered amount of drug that exits the device (i.e., the excluded the fraction in the nasal device) of the total collected amount.

A. Retinoid and Ratinoid Derivatives

The dry powder formulation contains a retinoid or a retinoid derivative, or a combination thereof. Generally, the retinoid and retinoid derivatives suitable for use in the dry powder formulation can serve as broad-spectrum antiviral agents by interrupting lipid metabolic reprogramming in the host cells. For example, one of the retinoid derivatives, tamibarotene, has broad-spectrum antiviral activity against various viruses, including influenza viruses and coronaviruses, such as severe acute respiratory syndrome coronaviruses (e.g., SARS-CoV-2), a Middle East respiratory syndrome coronavirus (“MERS-CoV”), and influenza A viruses (e.g., H1N1 virus), and a combination thereof.

Examples of retinoid and retinoid derivatives suitable for use in the dry powder formulation include, but are not limited to, retinol, tretinoin, isotretinoin, alitretinoin, etretinate, acitretin, adapalene, bexarotene, tazarotene, and tamibarotene, and a combination thereof. For example, the dry powder formulation contains tamibarotene for inhalation or intratracheal administration, and/or for intranasal administration into the lower and/or upper airways of a subject.

Generally, the amount of the retinoid or retinoid derivative, or the total amount of the retinoid and retinoid derivative in the dry powder formulation is in a range from about 10 wt % to about 80 wt %, from about 15 wt % to about 80 wt %, from about 20 wt % to about 80 wt %, from about 25 wt % to about 80 wt %, from about 30 wt % to about 80 wt %, from about 35 wt % to about 80 wt %, from about 40 wt % to about 80 wt %, from about 45 wt % to about 80 wt %, from about 50 wt % to about 80 wt %, from about 10 wt % to about 70 wt %, from about 15 wt % to about 70 wt %, from about 20 wt % to about 70 wt %, from about 25 wt % to about 70 wt %, from about 30 wt % to about 70 wt %, from about 35 wt % to about 70 wt %, from about 40 wt % to about 70 wt %, from about 45 wt % to about 70 wt %, from about 50 wt % to about 70 wt %, from about 10 wt % to about 60 wt %, from about 15 wt % to about 60 wt %, from about 20 wt % to about 60 wt %, from about 25 wt % to about 60 wt %, from about 30 wt % to about 60 wt %, from about 35 wt % to about 60 wt %, from about 40 wt % to about 60 wt %, from about 45 wt % to about 60 wt %, or from about 50 wt % to about 60 wt %, of the total weight of the retinoid and/or retinoid derivative and the β-cyclodextrin and/or β-cyclodextrin derivative. For example, the amount of the retinoid or retinoid derivative, or the total amount of the retinoid and retinoid derivative in the dry powder formulation is in a range from about 10 wt % to about 50 wt % of the total weight of the retinoid and/or retinoid derivative and the β-cyclodextrin and/or β-cyclodextrin derivative. Generally, the amount of the retinoid and retinoid derivative in the dry powder formulation can depend on the amount of cyclodextrin required based on the molecular weight, aqueous solubility, and potency of the drug. That is, when more than 20 wt % of cyclodextrin is needed, the maximum weight percentage of amount of the retinoid and retinoid derivative in the dry powder formulation will be less than 80. The term “total amount of the retinoid and retinoid derivative” refers to the sum of the weight of the retinoid and retinoid derivative relative to the sum of the weight of the retinoid and retinoid derivative and the β-cyclodextrin and/or β-cyclodextrin derivative in the dry powder formulation.

In some aspects, the dry powder formulation contains a retinoid derivative and the amount of the retinoid derivative is in a range from about 10 wt % to about 80 wt %, from about 15 wt % to about 80 wt %, from about 20 wt % to about 80 wt %, from about 25 wt % to about 80 wt %, from about 30 wt % to about 80 wt %, from about 35 wt % to about 80 wt %, from about 40 wt % to about 80 wt %, from about 45 wt % to about 80 wt %, from about 50 wt % to about 80 wt %, from about 10 wt % to about 70 wt %, from about 15 wt % to about 70 wt %, from about 20 wt % to about 70 wt %, from about 25 wt % to about 70 wt %, from about 30 wt % to about 70 wt %, from about 35 wt % to about 70 wt %, from about 40 wt % to about 70 wt %, from about 45 wt % to about 70 wt %, from about 50 wt % to about 70 wt %, from about 10 wt % to about 60 wt %, from about 15 wt % to about 60 wt %, from about 20 wt % to about 60 wt %, from about 25 wt % to about 60 wt %, from about 30 wt % to about 60 wt %, from about 35 wt % to about 60 wt %, from about 40 wt % to about 60 wt %, from about 45 wt % to about 60 wt %, or from about 50 wt % to about 60 wt % of the total weight of the retinoid derivative and the β-cyclodextrin and/or β-cyclodextrin derivative.

In some aspects, the dry powder formulation contains two or more retinoid derivatives and the amount of each retinoid derivative can be in a suitable range to provide a total amount of the retinoid derivatives in a range from about 10 wt % to about 80 wt %, from about 15 wt % to about 80 wt %, from about 20 wt % to about 80 wt %, from about 25 wt % to about 80 wt %, from about 30 wt % to about 80 wt %, from about 35 wt % to about 80 wt %, from about 40 wt % to about 80 wt %, from about 45 wt % to about 80 wt %, from about 50 wt % to about 80 wt %, from about 10 wt % to about 70 wt %, from about 15 wt % to about 70 wt %, from about 20 wt % to about 70 wt %, from about 25 wt % to about 70 wt %, from about 30 wt % to about 70 wt %, from about 35 wt % to about 70 wt %, from about 40 wt % to about 70 wt %, from about 45 wt % to about 70 wt %, from about 50 wt % to about 70 wt %, from about 10 wt % to about 60 wt %, from about 15 wt % to about 60 wt %, from about 20 wt % to about 60 wt %, from about 25 wt % to about 60 wt %, from about 30 wt % to about 60 wt %, from about 35 wt % to about 60 wt %, from about 40 wt % to about 60 wt %, from about 45 wt % to about 60 wt %, or from about 50 wt % to about 60 wt % of the total weight of the retinoid derivatives and the β-cyclodextrin and/or β-cyclodextrin derivative.

In some aspects, the dry powder formulation contains tamibarotene and the amount of tamibarotene is in a range from about 10 wt % to about 80 wt %, from about 15 wt % to about 80 wt %, from about 20 wt % to about 80 wt %, from about 25 wt % to about 80 wt %, from about 30 wt % to about 80 wt %, from about 35 wt % to about 80 wt %, from about 40 wt % to about 80 wt %, from about 45 wt % to about 80 wt %, from about 50 wt % to about 80 wt %, from about 10 wt % to about 70 wt %, from about 15 wt % to about 70 wt %, from about 20 wt % to about 70 wt %, from about 25 wt % to about 70 wt %, from about 30 wt % to about 70 wt %, from about 35 wt % to about 70 wt %, from about 40 wt % to about 70 wt %, from about 45 wt % to about 70 wt %, from about 50 wt % to about 70 wt %, from about 10 wt % to about 60 wt %, from about 15 wt % to about 60 wt %, from about 20 wt % to about 60 wt %, from about 25 wt % to about 60 wt %, from about 30 wt % to about 60 wt %, from about 35 wt % to about 60 wt %, from about 40 wt % to about 60 wt %, from about 45 wt % to about 60 wt %, or from about 50 wt % to about 60 wt % of the total weight of the tamibarotene and the β-cyclodextrin and/or β-cyclodextrin derivative.

B. β-Cyclodextrins and Derivatives Thereof

The dry powder formulation contains a β-cyclodextrin or a β-cyclodextrin derivative, or a combination thereof. β-cyclodextrin and β-cyclodextrin derivatives have a good safety profile for pulmonary delivery, and can function as solubilizer and stabilizer for the drug in the dry powder formulation.

In some aspects, the dry powder formulation contains a β-cyclodextrin. In some aspects, the dry powder formulation contains a β-cyclodextrin derivative, such as 2-hydroxypropyl-β-cyclodextrin (“HPPCD”), methyl-β-cyclodextrin (“MPCD”), or sulfobutylether β-cyclodextrin (“SBEPCD”), or a combination thereof. For example, the dry powder formulation contains HPPCD. In some aspects, the dry powder formulation contains a combination of a β-cyclodextrin and a β-cyclodextrin derivative. Other types of cyclodextrin, such as α-cyclodextrin and γ-cyclodextrin and their derivatives, can be used so long as they are compatible with the drug being used.

The β-cyclodextrin and/or β-cyclodextrin derivative in the dry powder formulation can improve the drug dissolution rate of the retinoid and/or retinoid derivatives, which can lead to faster drug adsorption in vivo. For example, following inhalation or intratracheal administration of the dry powder formulation to a subject, such as a mammal, the time to reach the maximum concentration of the retinoid and/or retinoid derivative in the lung of the subject is at least 2-time shorter, at least 3-time shorter, at least 4-time shorter, at least 5-time shorter, or at least 6-time shorter than the time to reach the maximum concentration of retinoid and/or retinoid derivative in a control. The control is the same species as the subject, which is administered with the same amount of retinoid and/or retinoid derivative as that in the dry powder formulation, but in an unformulated form, by intraperitoneal administration.

Generally, a β-cyclodextrin derivative can be obtained by chemical modification of the hydroxyl group(s) of β-cyclodextrin with an organic functional group, i.e., by substituting the hydrogen(s) of the hydroxyl group(s) with an organic functional group. For example, there are 21 hydroxyl groups on a β-cyclodextrin, which are 21 substitution sites for reaction with the organic functional group. A β-cyclodextrin derivative can have different degrees of substitution. The term “degree of substitution” refers to the number of hydroxyl groups that are modified with the organic functional group. For example, a degree of substitution of 6 means that 6 hydroxyl groups on the β-cyclodextrin are modified with an organic group.

The degree of substitution of the β-cyclodextrin derivative for use in the dry powder formulation can be in a range from 1 to 21, from 1 to 20, from 1 to 19, from 1 to 18, from 1 to 17, from 1 to 16, from 1 to 15, from 1 to 14, from 1 to 13, from 1 to 12, from 1 to 11, from 1 to 10, from 1 to 9, from 1 to 8, from 1 to 7, from 1 to 6, from 2 to 21, from 2 to 20, from 2 to 19, from 2 to 18, from 2 to 17, from 2 to 16, from 2 to 15, from 2 to 14, from 2 to 13, from 2 to 12, from 2 to 11, from 2 to 10, from 2 to 9, from 2 to 8, from 2 to 7, from 2 to 6, from 3 to 21, from 3 to 20, from 3 to 19, from 3 to 18, from 3 to 17, from 3 to 16, from 3 to 15, from 3 to 14, from 3 to 13, from 3 to 12, from 3 to 11, from 3 to 10, from 3 to 9, from 3 to 8, from 3 to 7, from 3 to 6, from 4 to 21, from 4 to 20, from 4 to 19, from 4 to 18, from 4 to 17, from 4 to 16, from 4 to 15, from 4 to 14, from 4 to 13, from 4 to 12, from 4 to 11, from 4 to 10, from 4 to 9, from 4 to 8, from 4 to 7, from 5 to 21, from 5 to 20, from 5 to 19, from 5 to 18, from 5 to 17, from 5 to 16, from 5 to 15, from 5 to 14, from 5 to 13, from 5 to 12, from 5 to 11, from 5 to 10, from 5 to 9, from 5 to 8, or from 5 to 7, such as from 6 to 7.

The β-cyclodextrin derivative for use in the dry powder formulation can have a molecular weight in a range from about 1000 g/mol to about 2500 g/mol, such as from about 1500 g/mol to about 2500 g/mol or from about 1000 g/mol to about 2000 g/mol. For example, the β-cyclodextrin derivative for use in the dry powder formulation is HPβCD, MβCD, or SBEβCD, or a combination thereof, and each of HPβCD, MβCD, and SBEβCD has a molecular weight in a range from about 1000 g/mol to about 2500 g/mol, such as from about 1500 g/mol to about 2500 g/mol or from about 1000 g/mol to about 2000 g/mol. The β-cyclodextrin derivative can be an anionic, a cationic, or a nonionic molecule.

1. Exemplary β-Cyclodextrin Derivatives

Exemplary β-cyclodextrin derivatives suitable for use in the dry powder formulation can have structures of Formula I.

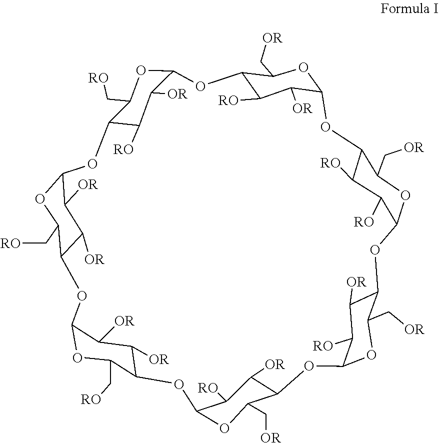

where each R is independently a hydrogen, an unsubstituted alkyl group, a substituted alkyl group, an unsubstituted heteroalkyl group, a substituted heteroalkyl group, an aldehyde group, or an acyl group optionally containing an unsubstituted alkyl group, a substituted alkyl group, an unsubstituted heteroalkyl group, a substituted heteroalkyl group, a halogen, an unsubstituted alkenyl group, a substituted alkenyl group, a hydroxyl group, an ether group, an amino group, a carboxylate group, or an amide group, and where the degree of substitution can be any one of the ranges described above, such as from 1 to 21, from 2 to 20, from 3 to 19, from 4 to 18, from 5 to 15, from 2 to 10, from 3 to 9, from 5 to 8, from 6 to 9, or from 6 to 8.

In some aspects of Formula I, the substituents for each substituted R are independently a sulfo group, a hydroxyl group, an unsubstituted alkyl group, a substituted alkyl group, an unsubstituted heteroalkyl group, a substituted heteroalkyl group, a halogen, an unsubstituted alkylene group, a substituted alkenyl group, a substituted alkenyl group, an ether group, an amino group, a carboxylate group, or an amide group.

In some aspects of Formula I, each R is independently a hydrogen, an unsubstituted alkyl group, a substituted alkyl group, or an acyl group optionally containing an unsubstituted alkyl group or a substituted alkyl group, and where the substituents for the substituted alky group are independently a sulfo group, a hydroxyl group, a carboxylate group, an amino group, or an amide group.

In some aspects of Formula I, each R is independently a hydrogen, an unsubstituted alkyl group, a substituted alkyl group, or an acyl group optionally containing an unsubstituted alkyl group or a substituted alkyl group (e.g., a formyl group, an acetyl group, a propionyl group, etc.), where the substituents for the substituted alkyl group are independently a sulfo group, a hydroxyl group, a carboxylate group, an amino group, or an amide group, and where the alkyl group (i.e., unsubstituted alkyl group or substituted alkyl group) is a linear C₁-C₃₀ alkyl, a branched C₄-C₃₀ alkyl, a cyclic C₃-C₃₀ alkyl, a linear C₁-C₃₀ alkyl or a branched C₄-C₃₀ alkyl, a linear C₁-C₃₀ alkyl or a cyclic C₃-C₃₀ alkyl, a branched C₄-C₃₀ alkyl or a cyclic C₃-C₃₀ alkyl, a linear C₁-C₂₀ alkyl, a branched C₄-C₂₀ alkyl, a cyclic C₃-C₂₀ alkyl, a linear C₁-C₂₀ alkyl or a branched C₄-C₂₀ alkyl, a branched C₄-C₂₀ alkyl or a cyclic C₃-C₂₀ alkyl, a linear C₁-C₂₀ alkyl or a cyclic C₃-C₂₀ alkyl, a linear C₁-C₁₀ alkyl, a branched C₄-C₁₀ alkyl, a cyclic C₃-C₁₀ alkyl, a linear C₁-C₁₀ alkyl or a branched C₄-C₁₀ alkyl, a branched C₄-C₁₀ alkyl or a cyclic C₃-C₁₀ alkyl, a linear C₁-C₁₀ alkyl or a cyclic C₃-C₁₀ alkyl, a linear C₁-C₆ alkyl, a branched C₄-C₆ alkyl, a cyclic C₃-C₆ alkyl, a linear C₁-C₆ alkyl or a branched C₄-C₆ alkyl, a branched C₄-C₆ alkyl or a cyclic C₃-C₆ alkyl, a linear C₁-C₆ alkyl or a cyclic C₃-C₆ alkyl, a linear C₁-C₅ alkyl, a branched C₄-C₅ alkyl, a cyclic C₃-C₅ alkyl, a linear C₁-C₅ alkyl or a branched C₄-C₅ alkyl, a branched C₄-C₅ alkyl or a cyclic C₃-C₅ alkyl, a linear C₁-C₅ alkyl or a cyclic C₃-C₅ alkyl, a linear C₁-C₄ alkyl, a branched C₄ alkyl, a cyclic C₃-C₄ alkyl, a linear C₁-C₄ alkyl or a cyclic C₃-C₄ alkyl, such as a linear C₁-C₅ alkyl group, a C₁-C₄ alkyl group, a C₁-C₃ alkyl group, or a C₁-C₂ alkyl group (e.g., a methyl group, an ethyl group, a propyl group, or a butyl group).

In some aspects of Formula I, each R is independently a hydrogen, a methyl group, an ethyl group, a propyl group, a butyl group, a hydroxyalkyl group (e.g., a hydroxymethyl group, a 1-hydroxyethyl group, a 2-hydroxyethyl group, a 1-hydroxypropyl group, a 2-hydroxypropyl group, a 3-hydroxypropyl group, a 1-hydroxybutyl group, a 2-hydroxybutyl group, a 3-hydroxybutyl group, a 4-hydroxybutyl group, etc.), a sulfoalkyl group (e.g., a sulfomethyl group, a 1-sulfoethyl group, a 2-sulfoethyl group, a 1-sulfopropyl group, a 2-sulfopropyl group, a 3-sulfopropyl group, a 1-sulfobutyl group, a 2-sulfobutyl group, a 3-sulfobutyl group, a 4-sulfobutyl group, etc.), a formyl group, an acetyl group, or a propionyl group, and where the degree of substitution is in a range from 1 to 12, from 2 to 15, from 2 to 12, from 2 to 10, from 4 to 10, from 5 to 8, from 6 to 9, or from 6 to 8.

In some aspects of Formula I, the β-cyclodextrin derivative is HPβCD, MβCD, or SBEβCD, and where the degree of substitution is in a range from 2 to 15, from 2 to 12, from 2 to 10, from 4 to 10, from 5 to 8, from 6 to 9, from 6 to 8, or from 6 to 7, such as CAVASOL®, Captisol®, and those described in Albers and Muller, “Cyclodextrin Derivatives in Pharmaceutics,” Critical Reviews in Therapeutic Drug Carrier Systems, 12(4):311-337 (1995).

In some aspects, the dry powder formulation can contain two or more β-cyclodextrin derivatives of different species. A different species can be a β-cyclodextrin derivative modified with the same functional group(s) with different degrees of substitution, a β-cyclodextrin derivative modified with different functional group(s) with the same degree of substitution, or a β-cyclodextrin derivatives modified with different functional group(s) and with a different degree of substitution.

For example, the dry powder formulation can contain two or more β-cyclodextrin derivatives, where each of the two or more β-cyclodextrin derivatives is modified with the same functional group with a different degree of substitution from the others. For example, the dry powder formulation can contain two or more β-cyclodextrin derivatives, where each of the two or more β-cyclodextrin derivatives is modified with a different functional group from the others and the degree of substitution is the same or different from the others.

2. Amount of β-Cyclodextrins and Derivatives Thereof

Generally, the amount of the β-cyclodextrin or β-cyclodextrin derivative, or the total amount of the β-cyclodextrin and β-cyclodextrin derivative in the formulation is at least 20 wt %, such as in a range from 20 wt % to 90 wt %, from 20 wt % to 85 wt %, from 20 wt % to 80 wt %, from 20 wt % to 75 wt %, from 20 wt % to 70 wt %, from 20 wt % to 65 wt %, from 20 wt % to 60 wt %, from 20 wt % to 55 wt %, from 20 wt % to 50 wt %, from 25 wt % to 90 wt %, from 25 wt % to 85 wt %, from 25 wt % to 80 wt %, from 25 wt % to 75 wt %, from 25 wt % to 70 wt %, from 25 wt % to 65 wt %, from 25 wt % to 60 wt %, from 25 wt % to 55 wt %, from 25 wt % to 50 wt %, from 30 wt % to 90 wt %, from 30 wt % to 85 wt %, from 30 wt % to 80 wt %, from 30 wt % to 75 wt %, from 30 wt % to 70 wt %, from 30 wt % to 65 wt %, from 30 wt % to 60 wt %, from 30 wt % to 55 wt %, from 30 wt % to 50 wt %, from 35 wt % to 90 wt %, from 35 wt % to 85 wt %, from 35 wt % to 80 wt %, from 35 wt % to 75 wt %, from 35 wt % to 70 wt %, from 35 wt % to 65 wt %, from 35 wt % to 60 wt %, from 35 wt % to 55 wt %, from 35 wt % to 50 wt %, from 40 wt % to 90 wt %, from 40 wt % to 85 wt %, from 40 wt % to 80 wt %, from 40 wt % to 75 wt %, from 40 wt % to 70 wt %, from 40 wt % to 65 wt %, from 40 wt % to 60 wt %, from 40 wt % to 55 wt %, from 40 wt % to 50 wt %, from 45 wt % to 90 wt %, from 45 wt % to 85 wt %, from 45 wt % to 80 wt %, from 45 wt % to 75 wt %, from 45 wt % to 70 wt %, from 45 wt % to 65 wt %, from 45 wt % to 60 wt %, from 45 wt % to 55 wt %, from 50 wt % to 90 wt %, from 50 wt % to 85 wt %, from 50 wt % to 80 wt %, from 50 wt % to 75 wt %, from 50 wt % to 70 wt %, from 50 wt % to 65 wt %, or from 50 wt % to 60 wt % of the total weight of the retinoid and/or retinoid derivative and the β-cyclodextrin and/or β-cyclodextrin derivative. For example, the amount of the β-cyclodextrin or β-cyclodextrin derivative, or the total amount of the β-cyclodextrin and β-cyclodextrin derivative in the formulation can be in a range from 20 wt % to 90 wt % of the total weight of the retinoid and/or retinoid derivative and the β-cyclodextrin and/or β-cyclodextrin derivative. The term “total amount of the β-cyclodextrin and β-cyclodextrin derivative” refers to the sum of the weight of the β-cyclodextrin and β-cyclodextrin derivative relative to the sum of the weight of the retinoid and/or retinoid derivative and the β-cyclodextrin and β-cyclodextrin derivative in the dry powder formulation.

In some aspects, the dry powder formulation contains HPβCD with a single degree of substitution or different degrees of substitution and the amount of HPβCD with a single degree of substitution or the total amount of HPβCD with different degrees of substitution is at least 20 wt %, in a range from 20 wt % to 90 wt %, from 20 wt % to 85 wt %, from 20 wt % to 80 wt %, from 20 wt % to 75 wt %, from 20 wt % to 70 wt %, from 20 wt % to 65 wt %, from 20 wt % to 60 wt %, from 20 wt % to 55 wt %, from 20 wt % to 50 wt %, from 25 wt % to 90 wt %, from 25 wt % to 85 wt %, from 25 wt % to 80 wt %, from 25 wt % to 75 wt %, from 25 wt % to 70 wt %, from 25 wt % to 65 wt %, from 25 wt % to 60 wt %, from 25 wt % to 55 wt %, from 25 wt % to 50 wt %, from 30 wt % to 90 wt %, from 30 wt % to 85 wt %, from 30 wt % to 80 wt %, from 30 wt % to 75 wt %, from 30 wt % to 70 wt %, from 30 wt % to 65 wt %, from 30 wt % to 60 wt %, from 30 wt % to 55 wt %, from 30 wt % to 50 wt %, from 35 wt % to 90 wt %, from 35 wt % to 85 wt %, from 35 wt % to 80 wt %, from 35 wt % to 75 wt %, from 35 wt % to 70 wt %, from 35 wt % to 65 wt %, from 35 wt % to 60 wt %, from 35 wt % to 55 wt %, from 35 wt % to 50 wt %, from 40 wt % to 90 wt %, from 40 wt % to 85 wt %, from 40 wt % to 80 wt %, from 40 wt % to 75 wt %, from 40 wt % to 70 wt %, from 40 wt % to 65 wt %, from 40 wt % to 60 wt %, from 40 wt % to 55 wt %, from 40 wt % to 50 wt %, from 45 wt % to 90 wt %, from 45 wt % to 85 wt %, from 45 wt % to 80 wt %, from 45 wt % to 75 wt %, from 45 wt % to 70 wt %, from 45 wt % to 65 wt %, from 45 wt % to 60 wt %, from 45 wt % to 55 wt %, from 50 wt % to 90 wt %, from 50 wt % to 85 wt %, from 50 wt % to 80 wt %, from 50 wt % to 75 wt %, from 50 wt % to 70 wt %, from 50 wt % to 65 wt %, or from 50 wt % to 60 wt % of the total weight of the retinoid and/or retinoid derivative and the HPβCD. When the dry powder formulation contains HPβCD of different degrees of substitution, the amount of each species can be in a suitable range to provide the above-described ranges.

In some aspects, the dry powder formulation contains MβCD with a single degree of substitution or different degrees of substitution and the amount of MβCD with a single degree of substitution or the total amount of MβCD with different degrees of substitution can be in any of the above-described concentration ranges for HPβCD. When the dry powder formulation contains MβCD of different degrees of substitution, the amount of each species can be in a suitable range to provide the above-described ranges.

In some aspects, the dry powder formulation contains SBEPCD with a single degree of substitution or different degrees of substitution and the amount of SBEPCD with a single degree of substitution or the total amount of SBEPCD with different degrees of substitution can be in any of the above-described ranges for HPβCD. When the dry powder formulation contains SBEPCD of different degrees of substitution, the amount of each species can be in a suitable range to provide the above-described ranges.

C. Pharmaceutically Acceptable Excipients

The dry powder formulation may contain a pharmaceutically acceptable excipient, optionally more than one pharmaceutically acceptable excipient. In some aspects, the dry powder formulation does not contain any additional pharmaceutically acceptable excipients.

Exemplary pharmaceutically acceptable excipients that can be used in the dry powder formulation include, but are not limited to, amino acids, peptides, lipids (e.g., fatty acids, fatty acid esters, steroids), proteins, chelating agents (e.g., EDTA), salts, taste masking agents, cations, non-biological or biological polymers, and additional sugars, and a combination thereof. Preferred excipients include sugars or sugar alcohols such as lactose, trehalose, mannitol. Examples of suitable pharmaceutically acceptable excipients that can be used in the dry powder formulation are described in Kibbe, et al., “Handbook of Pharmaceutical Excipients,” 3^{rd} edition, 2000. In some aspects, the dry powder formulation does not contain any additional sugars.

Examples of suitable amino acids that may be include in the dry powder formulation include, but are not limited to, alanine, glycine, arginine, histidine, glutamate, asparagine, cysteine, lucine, lysine, isoleucine, valine, tryptophan, methionine, proline, phenylalanine, tyrosine, citrulline, L-aspartyl-L-phenylalanine-methyl ester (aspartame), trimethylammonium acetate (betaine), etc.

Examples of suitable protein excipients that may be included in the dry powder formulation include, but are not limited to, albumin (of human or recombinant origin), gelatin, casein, hemoglobin, etc.

Examples of suitable polymers that may be included in the dry powder formulation include, but are not limited to, polyvinyl pyrrolidone, derivatized celluloses, such as hydroxymethyl, hydroxyethyl, hydroxypropyl ethylcellulose, polyethylene glycol, and polypropylene glycol.

Examples of additional sugars that may be included in the dry powder formulation can be a mono-, di-, oligo-, or polysaccharide, or a combination thereof. Examples of monosaccharides are fructose, maltose, galactose, glucose, D-mannose, sorbose, and the like. Examples of disaccharides are lactose, saccharose, trehalose, cellobiose, etc. Examples of sugar alcohols are mannitol, xylitol, maltitol, galactitol, arabinitol, adonitol, lactitol, sorbitol (glucitol), pyranosylsorbitol, inositol, myoinositol, etc. Examples of oligosaccharide are other types of cyclodextrin (e.g., α-cyclodextrin, γ-cyclodextrin, and their derivatives thereof), maltodextrin, and pectins.

In some aspects, the dry powder formulation does not contain any additional sugars, such as mannitol, trehalose, 1,4 O-linked saccharose or 1,4 O-linked saccharose derivatives, or dexran, or a combination thereof. In some aspects, the dry powder formulation does not contain mannitol or trehalose, or a combination of mannitol and trehalose. In some aspects, the dry powder formulation does not contain 1,4 O-linked saccharose or 1,4 O-linked saccharose derivatives, or a combination thereof. In some aspects, the dry powder formulation does not contain dexran. In some aspects, the dry powder formulation does not contain any one of mannitol, trehalose, 1,4 O-linked saccharose, 1,4 O-linked saccharose derivatives, and dexran.

Examples of salts that may be included in the dry powder formulation are inorganic salts such as chloride, sulphate, phosphate, diphosphate, hydrobromide, and nitrate salts and organic salts such as malate, maleate, fumarate, tartrate. Succinate, ethylsuccinate, citrate, acetate, lactate, methaneSulphonate, benzoate, ascorbate, para-toluenesulphonate, palmoate, salicylate, stearate, estolate, gluceptate, and lactobionate salts. The salts may simultaneously contain pharmaceutically acceptable cations, such as sodium, potassium, calcium, aluminium, lithium, and ammonium.

The amount of the pharmaceutical acceptable excipient, or the total amount of two or more pharmaceutically acceptable excipients may be in a range from 0.1 wt % to 20 wt %, from, from 0.1 wt % to 15 wt %, from 1 wt % to 12 wt %, from 1 wt % to 10 wt %, from 1 wt % and 15 wt %, from 2 wt % to 20 wt %, from 2 wt % to 15 wt %, from 2 wt % to 10 wt %, from 3 wt % to 20 wt %, from 3 wt % to 15 wt %, or from 3 wt % to 10 wt % of the dry powder formulation. The term “total amount of the two or more pharmaceutically acceptable excipients” refers to the sum of the weight of the two or more pharmaceutically acceptable excipients relative to the total weight of the dry powder formulation.

D. Additional Active Agents

The dry powder formulation may contain an additional active agent, optionally more than one additional active agent. The additional active agents that can be included in the dry powder formulation may be therapeutic, nutritional, prophylactic, or diagnostic agents, or a combination thereof, such as anti-inflammatory agents and antiviral agents.

Examples of anti-inflammatory and antiviral agents that can be included in the dry powder formulation include, but are not limited to, triamcinolone acetonide, fluocinolone acetonide, prednisolone, dexamethasone, loteprendol, fluorometholone, ketorolac, nepafenac, diclofenac, ribavirin, favipiravir, remdesivir, clofazimine, N-(p-amylcinnamoyl)anthranilic acid, and a combination thereof.

The amount of the additional active agents needed will vary from subject to subject according to their need.

E. Delivery Systems

Systems for delivering the dry powder formulations are disclosed.

In some aspects, the system includes an inhaler and the dry powder formulation. The dry powder formulation may be in a unit dosage form that contains a single unit does of the retinoid and/or retinoid derivative. Alternatively, the dry powder formulation may contain multiple doses of the retinoid and/or retinoid derivative.

In some aspects, the system includes a nasal device and a solution or suspension formed by the dry powder formulation and a suitable solvent. The dry powder formulation used for forming the solution or suspension may be in a unit dosage form that contains a single unit does of the retinoid and/or retinoid derivative. Alternatively, the dry powder formulation used for forming the solution or suspension may contain multiple doses of the retinoid and/or retinoid derivative.

1. Unit Dosage Form

In some aspects, the dry powder formulation is prepackaged in a capsule or replaceable set and then loaded in an inhaler is in a unit dosage form, i.e., containing a single unit does of the retinoid and/or retinoid derivatives to be delivered to the lung(s) of a subject.

In some aspects, for intranasal use, the dry powder formulation can be formulated into an aqueous solution or suspension using a suitable solvent prior to use and then the aqueous solution or suspension can be loaded in a nasal device as a unit dosage form, i.e., containing a single unit does of the retinoid and/or retinoid derivatives to be delivered to the upper respiratory tract of a subject. Examples of solvent suitable for forming the aqueous solution or suspension for intranasal administration as drops or as a spray include, but are not limited to, distilled water, de-ionized water, pure or ultrapure water, saline, phosphate-buffered saline (PBS), Ringer's solution, and isotonic sodium chloride, and a combination thereof. Generally, the choice of solvent depends on the device used for administration. For example, the mucosal atomizer devices in common use are suitable for aqueous solutions and suspensions. Ultrapure water is the preferred solvent for low concentrations. Suspension may be formed when the concentration is higher than its solubility.

Such aqueous solutions or suspensions may be isotonic relative to nasal secretions and of about the same pH, ranging e.g., from about pH 4.0 to about pH 7.4 or, from pH 6.0 to pH 7.0. Buffers should be physiologically compatible and include, simply by way of example, phosphate buffers. One skilled in the art can readily determine a suitable saline content and pH for an innocuous aqueous solution for nasal and/or upper respiratory administration. Aqueous suspensions may include suspending agents such as cellulose derivatives, sodium alginate, polyvinyl-pyrrolidone and gum tragacanth, and a wetting agent such as lecithin. Suitable preservatives for aqueous suspensions include ethyl and n-propyl p-hydroxybenzoate.

A suitable unit dose of the retinoid and/or retinoid derivatives in the unit dosage form of the dry powder formulation can range from about 0.1 mg to about 50 mg, from about 0.5 to about 50 mg, or from about 1 to about 50 mg. For example, a suitable unit dose of the retinoid and/or retinoid derivatives prepackaged in a capsule is in a range from about 0.1 mg to about 50 mg, from about 0.5 to about 50 mg, or from about 1 to about 50 mg.

Alternatively, the dry powder formulation or the solution or suspension formed by the dry powder formulation can contain multiple unit doses of the retinoid and/or retinoid derivatives that can be loaded in the reservoir of a metered inhaler or nasal device such that a single dose may be delivered to the subject per administration.

Treatment regimens utilizing retinoid and/or retinoid derivatives can include administration of from about 0.1 mg to about 100 mg of the retinoid and/or retinoid derivatives per kilogram body weight of the recipient per day in a single unit dose or multiple unit doses (such as two, three, four, five, or six or more unit doses at appropriate intervals throughout the day).

2. Inhalers
a. Dry Powder Inhalers

Typically, suitable inhalers that can be used with the dry powder formulation are dry powder inhalers (“DPIs”). The dry powder formulation may be pre-packaged in a capsule or a replaceable set and then loaded in the inhaler. Alternatively, the dry powder formulation may be directly loaded in the reservoir of the inhaler.

DPIs are breath actuated, thus the problem of coordinated inspiration with actuation, as in the case of pMDIs, is avoided. Exemplary dry powder inhaler types include single capsule unit dose in an inhaler, single disposable unit dose in the inhaler, multiple unit doses in a replaceable set in an inhaler, and multiple unit doses in a reservoir in an inhaler.

The dry powder inhaler may carry one or more capsules, each capsule containing a unit dose of the dry powder formulation in unit dosage form. For example, the DPI may be a single unit dose DPI containing one capsule loaded with one unit dose of the dry powder formulation in unit dosage form, where the capsule may be repeatedly loaded or disposable. Alternatively, the DPI may be a multiple unit doses DPI containing two or more capsules where each of the two or more capsules is loaded with one unit dose of the dry powder formulation in unit dosage form and the capsules may be repeatedly loaded or disposable.

The dry powder inhalers may contain a replaceable set with multiple doses of the dry powder formulation in unit dosage form, such as a replaceable blister package, cartridge, strip, or wheel. For example, the DPI may be a multiple unit doses DPI containing a foil-foil blister prepackaged with several unit doses of the dry powder formulation in unit dosage form, where each unit dose is spatially separated from the other unit doses (i.e., discrete unit doses).

The dry powder inhalers may contain a reservoir with a capacity for holding multiple unit doses of the dry powder formulation in unit dosage form. For example, the DPI may be a multiple unit doses DPI containing a pre-metered cartridge containing multiple unit doses of the dry powder formulation in unit dosage form. In some aspects, the DPI may be a multiple unit doses DPI containing a dry powder formulation that contains multiple unit doses of the retinoid and/or retinoid derivative in a reservoir. For these DPIs, it may include a pre-metered valve such that one unit dose can be delivered to the subject per administration.

Suitable DPIs for use with the dry powder formulations may be those described in U.S. Pat. No. 7,305,986 and U.S. Patent Application Publication No. 2004/0182387. Exemplary commercially available multi-dose DPIs suitable for use with the dry powder formulation include, but are not limited to, DISKUS® (Glaxo Group Limited Corp, Brentford, Middlesex United Kingdom), DISKHALER® (Glaxo Group Limited Corp, Brentford, Middlesex United Kingdom), GEMINI® (GSK, also described in WO 05/14089), GYROHALER® (Vectura, also described in WO 05/37353), PROHALER® (Valois, also described in WO 03/77979) and TWISTHALER® (Merck, also described in WO 93/00123, WO 94/14492, and WO 97/30743). Exemplary commercially available single dose DPIs suitable for use with the dry powder formulation include, but are not limited to, AEROLIZER® (Novartis Ag Corporation Switzerland, Basel, Switzerland) and BREEZHALER® (Novartis Ag Corporation Switzerland, Basel, Switzerland). Other exemplary DPIs suitable for use with the dry powder formulation include, but are not limited to high-resistance Osmohale™ inhaler, Breezhaler®, HANDIHALER® (Boehringer Ingelheim Pharma KG, Ingelheim am Rhein, Fed Rep Germany), DIRECT HALER@ (Direct-Haler A/S Corp Denmark, Odense Sv Denmark), ELLIPTA® (Glaxo Group Limited Corp, Brentford, Middlesex United Kingdom), TURBUHALER® (Astra Aktiebolag Corp., Sodertalie Sweden), EASYHALER® (Orion Corporation, Espoo Finland), and Nexthaler (Lavorini et al. Multidisciplinary Respiratory Medicine, 12:11 (2017)).

3. Nasal Devices

Suitable nasal devices that can be used with a solution or suspension formed by the dry powder formulation to deliver retinoid and/or retinoid derivatives as a drop or spray include, but are not limited to, a metered dose spray pump, an atomizer, a syringe, a bulb, a canister, a pressurized container, a spray can, or a nebulize. The dry powder formulation and solvent may be pre-packaged in separate containers, mixed to prepare a solution or suspension prior to use, and then loaded in the nasal device. Alternatively, the dry powder formulation may be directly provided as a solution or suspension prepackaged in a container for loading into the nasal device and pre-loaded in the nasal device.

F. Particle Properties

The aerosolisation performance of the dry powder formulation can be evaluated by the properties of particles of the dry powder formulation, such as mass median aerodynamic diameter (“MMAD”), volumetric mean diameter (“VMD”), fine particle fraction (“FPF”), fraction of particles>9 μm (for intranasal administration), and emitted fraction (“EF”). The term “fine particle fraction” or “FPF” refers to the weight percentage of particles with MMAD less than 5 μm relative to the total weight of particles in the formulation. The term “fraction of particles>9 μm” or “FP9” refers to the weight percentage of particles with MMAD larger than 9 μm relative to the total weight of particles in the formulation. The term “EF” refers to the fraction of powder that exits the inhale after a dispersion event, expressed as the ratio of the dose delivered by an inhaler to the nominal dose, i.e., the mass of powder per unit dose placed into the inhaler prior to dispersion. The term “volumetric mean diameter” or “VMD” refers to the equivalent spherical volume diameters at 90% cumulative volume of the particles.

MMAD, VMD, and FPF of the particles in the dry powder formulation may be determined using methods known in the art. These include dynamic light scattering, aerodynamic particle sizing, light microscopy, laser diffractometer, scanning electron microscopy (“SEM”), reduced Andersen Cascade Impactor (“ACI”), and/or cryo-transmission electron microscopy (“cryo-TEM”). For example, VMD may be measured by laser diffractometer and/or SEM. For example, MMAD and FPF may be measure by dispersing powder by a high-resistance Osmohale™ inhaler and the powder is evaluated by a Next Generation Impactor (NGI) operated at, for example, 45 L/min for up to 5.4 s, 60 L/min for up to 4 s, or 90 L/min for up to 2.7 s. For example, MMAD and FPF may be measure by dispersing powder by a Breezhaler® and the powder is evaluated by a Next Generation Impactor (NGI) operated at 90 L/min for about 2.7 s. Generally, the NGI and time should be adjusted based on the accepted volume. Ph. Eur suggests 4 litres of air; FDA and USP suggests 2 litres of air, so a range between 2 to 4 litres is acceptable and preferred.

In some aspects, the particles of the dry powder formulation have favorable aerodynamic properties for effective lung deposition and retention. For example, the particles of the dry powder formulation have a MMAD<5 μm, <4 μm, <3.5 μm, <3 μm, <2.5 μm, or <2 μm; a FPF>40%, >45%, >50%, >55%, >60%, or >65% in cascade impactor study; and/or an EF>65%, >70%, >75%, >80%, >85%, >90%, >92%, or >95%.

In some aspects, the particles of the dry powder formulation have favorable aerodynamic properties for effective deposition and retention in the upper respiratory tract by intranasal administration. For example, the particles of the dry powder formulation have a MMAD>9 μm, >9.5 μm, >10 μm, or >10.5 μm; a FP9>40%, >45%, >50%, >55%, or >60% in ACI study; and/or an EF>85%, >90%, or >95%.

Additional particle properties, such as dissolution rate and bioavailability in the lung and plasma can be determined by dissolution study and pharmacokinetic study, respectively. Specific exemplary measurement conditions are described in the Examples below.

G. Exemplary Formulations

An exemplary dry powder formulation contains spray-dried or spray-freeze-dried particles containing HPβCD and tamibarotene (structure shown below), where the weight ratio of HPβCD to tamibarotene is in a range from 1:4 to 9:1, from 1:3 to 9:1, from 1:2 to 9:1, or from 1:1 to 9:1.

For these dry powder formulations, the HPβCD may have a degree of substitution in a range from 2 to 15, from 2 to 12, from 2 to 10, from 4 to 10, from 5 to 8, from 6 to 9, from 6 to 8, or from 6 to 7. For example, the HPβCD has the following structure.

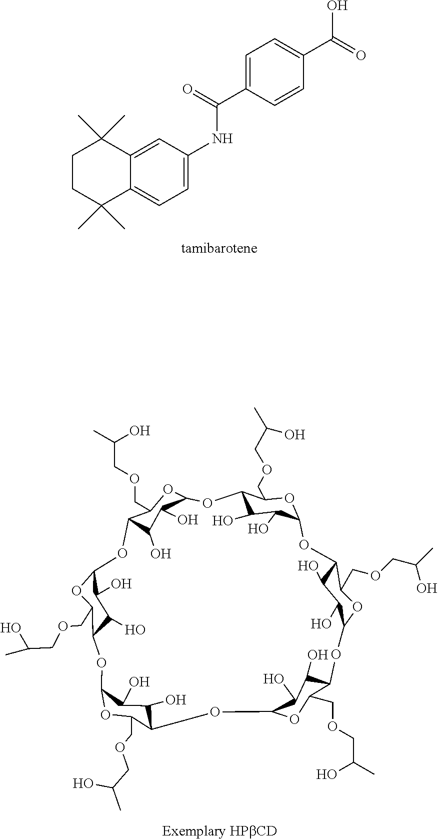 [SMILES: CC1(C)CCC(C)(C)c2cc(NC(=O)c3ccc(C(=O)O)cc3)ccc21 ; CC(O)COCC1O[C@@H]2O[C@@H]3C(COCC(C)O)O[C@@H](O[C@@H]4C(COCC(C)O)O[C@H](O[C@@H]5C(COCC(C)O)O[C@@H](O[C@@H]6C(COCC(C)O)O[C@@H](O[C@@H]7C(COCC(C)O)O[C@H](O[C@H]1C(O)C2O)C(O)C7O)C(O)C6O)C(O)C5O)C(O)C4O)C(O)C3O]

These dry powder formulations may be in a unit dosage form prepackaged in a capsule or a replaceable set and then loaded in a dry powder inhaler, or formulated into an aqueous solution or suspension prior to use and then loaded in a nasal device. Alternatively, these dry powder formulations or solution or suspensions formed by these dry powder formulations may contain multiple doses of tamibarotene and can be loaded in the reservoir of a dry powder inhaler or a nasal device.

The particles of these dry powder formulations can have a MMAD<5 μm, <4 μm, <3.5 μm, <3 μm, <2.5 μm, or <2 μm; a FPF>40%, >45%, >50%, >55%, >60%, or >65% in cascade impactor study; and/or an EF>65%, >70%, >75%, >80%, >85%, >90%, >92%, or >95%.

Alternatively, the particles of these dry powder formulations can have a MMAD>9 μm, >9.5 μm, >10 μm, or >10.5 μm; a FP9>40%, >45%, >50%, >55%, or >60% in ACI study; and/or an EF>85%, >90%, or >95%.

These dry powder formulations show faster tamibarotene adsorption and improved tamibarotene bioavailability in vivo. For example, following inhalation or intratracheal administration of the dry powder formulation to a subject, such as a mammal, the maximum concentration (“Cₘₐₓ”) of the tamibarotene delivered to the lung of the subject is at least 10-time higher, at least 15-time higher, at least 20-time higher, at least 25-time higher, at least 30-time higher, or at least 35-time higher than the maximum concentration of tamibarotene delivered to the lung of a control; the Cₘₐₓ of tamibarotene in the lung of the subject is at least 40-time higher or at least 45-time higher than the EC₅₀ for SARS-CoV-2, at least 800-time higher than the EC₅₀ for MERS-CoV, and/or at least 150-time higher than the EC₅₀ for H1N1. Additionally, the time to reach the maximum concentration of tamibarotene (“Tₘₐₓ”) in the lung of the subject is at least 2-time shorter, at least 3-time shorter, at least 4-time shorter, at least 5-time shorter, or at least 6-time shorter than the time to reach the maximum concentration of tamibarotene in the control. The control is the same species as the subject, which is administered with the same amount of tamibarotene as that in the dry powder formulation, but in an unformulated form, by intraperitoneal administration. More specific exemplary Cₘₐₓ and Tₘₐₓ values measured by in vivo pharmacokinetic studies are described in the Examples below.

More specific exemplary dry powder formulations are described in the Examples.

### III. Methods of Making the Dry Powder Formulations

Methods of making the dry powder formulations are disclosed. Generally, the method includes (i) mixing a retinoid and/or a retinoid derivative and a β-cyclodextrin and/or a β-cyclodextrin derivative, and optionally a pharmaceutically acceptable excipient and/or an additional active agent, in a solvent to form a liquid feed; and (ii) spray-drying or spray-freeze drying the liquid feed to form particles containing the retinoid and/or retinoid derivative and the β-cyclodextrin and/or β-cyclodextrin derivative, and optionally the pharmaceutically acceptable excipient and/or the additional active agent.

Typically, the production yield of particles following step (ii) is at least 40 wt %, at least 50 wt %, at least 60 wt %, at least 65 wt %, at least 70 wt %, at least 75 wt %, at least 80 wt %, at least 85 wt %, at least 90 wt %, in a range from about 40 wt % to about 95 wt %, from about 55 wt % to about 95 wt %, from about 55 wt % to about 95 wt %, from about 60 wt % to about 95 wt %, or from about 65 wt % to about 95 wt %. The yield of particles is the weight of particles produced relative to the total weight of ingredients in the liquid feed. The “total weight of ingredients” refers to the total weight of the retinoid and/or retinoid derivative and the β-cyclodextrin and/or β-cyclodextrin derivative, and optionally also includes the weight of the pharmaceutically acceptable excipient, additional active agent, and/or salts in the solvent.

The particles formed in step (ii) by spray-drying or spray-freeze drying have favorable aerodynamic properties for effective lung or upper respiratory tract deposition and retention, as described above. Additionally, when solvent sublimed in the freeze drying process, the frozen crystal transit from solid to gas phase, leaving pores in the resulting solid particles. The porous structure can increase the surface area of the particles to allow rapid dissolution of the drugs.

A. Forming Liquid Feed

Generally, prior to spray-drying or spray-freeze-drying, a liquid feed is prepared by mixing the retinoid and/or retinoid derivative and the β-cyclodextrin and/or β-cyclodextrin derivative, and optionally a pharmaceutically acceptable excipient and/or an additional active agent in a solvent. Typically, the solvent is an aqueous solvent. In some aspects, the liquid feed only contains the retinoid and/or retinoid derivative and the β-cyclodextrin and/or β-cyclodextrin derivative in the aqueous solvent.

The complex formed by retinoid and/or retinoid derivative and the β-cyclodextrin and/or β-cyclodextrin derivative can be soluble in the aqueous solvent. For example, the complex formed by retinoid and/or retinoid derivative and the β-cyclodextrin and/or β-cyclodextrin derivative have a solubility of at least about 1.5 g/100 mL of the aqueous solvent at room temperature (R.T.), i.e., a temperature between about 20° C. and about 25° C. under atmospheric pressure. For retinoids and retinoid derivatives that are less soluble in aqueous solutions (such as tamibarotene), and for which the cyclodextrin cannot provide sufficient solubilization effect, a different solvent can be used. For example, tert-butyl alcohol (TBA) can be used (it is useful because it has a high freezing point at 25° C. for easy sublimation). TBA can be used as the solvent to dissolve tamibarotene at first. Then the tamibarotene/TBA solution can be mixed with the cyclodextrin aqueous solution, and the mixed solution can be stirred for 4 h to allow sufficient interaction prior to spray freeze drying. In spray drying, TBA can be replaced by ethanol (it has a low boiling point for easy evaporation). Organic solvent can be used in feed solutions as long as it can be completely removed (or removed to below toxic limit as specified by ICH) after drying. When a pharmaceutically acceptable excipient and/or an additional active agent are mixed in the solvent, they may be soluble or suspended in the solvent. For example, the pharmaceutically acceptable excipient and/or additional active agent can be at least as soluble as the retinoid and/or retinoid derivative in the solvent or insoluble in the solvent.

Suitable solvents for preparing the liquid feed include, but are not limited to, water and aqueous buffers, such as sodium phosphate, potassium phosphate, sodium acetate, potassium acetate, sodium citrate, potassium citrate, sodium succinate, potassium succinate, and ammonium bicarbonate and carbonate, and a combination thereof. Generally, aqueous buffers that are suitable for preparing the liquid feed have a molarity in a range from about 1 mM to about 2 M, from about 2 mM to about 1 M, from about 10 mM to about 0.5 M, or from 50 to 200 mM and have a pH in a range from about 1 to about 10, from about 3 to about 8, or from about 5 to about 7.

The concentration of solute in the solvent, i.e., the total amount of retinoid and/or retinoid derivative and the β-cyclodextrin and/or β-cyclodextrin derivative in the solvent, can be in a range from about 0.5 mg/mL to about 200 mg/mL, from about 1 mg/mL to about 200 mg/mL, from about 5 mg/mL to about 200 mg/mL, from about 10 mg/mL to about 200 mg/mL, from about 1 mg/mL to about 200 mg/mL, from about 1 mg/mL to about 200 mg/mL, from about 1 mg/mL to about 200 mg/mL, from about 1 mg/mL to about 100 mg/mL, or from about 10 mg/mL to about 100 mg/mL, such as about 50 mg/mL for spray-drying or for spray-freeze-drying.

The liquid feed prepared in step (i) is then subject to spray-drying or spray-freeze drying process.

B. Spray-Drying

Spray-drying is a process of producing a dry powder containing particles from a liquid or a dispersion in a liquid by rapidly drying with a hot gas. This process can rapidly produce particles for inhalation (i.e., on the order of milliseconds) with controlled particle size, morphology, density, and surface composition.

Generally, in step (ii), the liquid feed prepared in step (i) may be spray-dried to form the dry powder formulation disclosed herein. The spray-drying process may be carried out using conventional equipment used to prepare spray dried particles for use in pharmaceuticals that are administered by inhalation. Exemplary commercially available spray-dryers include those manufactured by Buchi Ltd., Niro Corp, Bichi, Niro Yamato, Okawara, Kakoki.

Typically, the liquid feed is sprayed through a nozzle, such as a two-fluid nozzle or an ultrasound nozzle, into a current of warm filtered air that evaporates the solvent and conveys the dried product to a collector. The spent air is then exhausted with the solvent. Operating conditions of the spray-dryer such as inlet and outlet temperature, feed rate, atomization pressure, flow rate of the drying air, and nozzle configuration can be adjusted in order to produce the required particle size, moisture content, and production yield of the resulting dry particles. The selection of appropriate apparatus and processing conditions are within the purview of a skilled artisan in view of the teachings herein.

Exemplary settings for the spray-drying step are as follows: an air inlet temperature between about 60° C. and about 220° C., between about 80° C. and about 220° C., between about 60° C. and about 200° C., between about 60° C. and about 180° C., between about 80° C. and about 200° C., between about 80° C. and about 180° C., between about 80° C. and 150° C., or between about 90° C. and 120° C.; an air outlet temperature between about 40° C. to about 120° C., between about 50° C. and 100° C., or between about 50° C. and 80° C.; a feed rate between about 0.1 mL/min to about 30 mL/min, between about 0.1 mL/min to about 25 mL/min, between about 0.1 mL/min to about 20 mL/min, between about 0.1 mL/min to about 15 mL/min, between about 0.5 mL/min to about 30 mL/min, between about 0.5 mL/min to about 25 mL/min, between about 0.5 mL/min to about 20 mL/min, between about 0.5 mL/min to about 15 mL/min, between about 1 mL/min to about 30 mL/min, between about 1 mL/min to about 25 mL/min, between about 1 mL/min to about 20 mL/min, between about 1 mL/min to about 15 mL/min, between about 1.5 mL/min to about 15 mL/min, between about 1 mL/min to about 10 mL/min, or between about 1 mL/min to about 5 mL/min; an air flow rate between about 100 L/h to about 1000 L/h, between about 200 L/h to about 1000 L/h, between about 300 L/h to about 1000 L/h, between about 400 L/h to about 1000 L/h, between about 500 L/h to about 1000 L/h, between about 500 L/h to about 900 L/h, between about 600 L/h to about 800 L/h, or between about 650 L/h to about 800 L/h; and optionally an aspiration rate of up to 50%, up to 60%, up to 70%, up to 80%, up to 90%, or up to 100%, for example, an aspiration rate of 100%, i.e., about 35 m³/h, using a Buchi B290 spray dryer. These settings will, however, vary depending on the type of equipment used, and the nature of the solvent system employed. In any event, the use of these and similar methods allow formation of particles with diameters appropriate for aerosol deposition into the respiratory tract, such as the lung or the upper respiratory tract.

An exemplary spray-drying process uses a Büchi B-290 spray drier and have the following settings: an air inlet temperature between about 90° C. and 120° C., such as about 100° C.; an air outlet temperature between about 50° C. and 80° C., such as between 62° C. and 67° C.; a feed rate between about 0.1 mL/min and 30 mL/min, such as about 1.5 mL/min, an air flow rate between about 550 L/h to about 700 L/h, such as about 601 L/h, and an aspiration rate of 100%, i.e., about 35 m³/h, where the production yield of particles following spray drying is at least 40 wt %, such as between about 40 wt % and about 95 wt %.

C. Spray-Freeze-Drying

Spray-freeze-drying is a process similar to spray drying in that a liquid feed containing the retinoid and/or retinoid derivative and the β-cyclodextrin and/or β-cyclodextrin derivative, and optionally the pharmaceutically acceptable excipient and/or additional active agent is introduced via a nozzle (e.g., a two-fluid nozzle or an ultrasound nozzle), or a spinning disk into a cold fluid to atomize the liquid feed to form fine droplets. The cold fluid, either a liquid or a gas, is at a temperature below the freezing point of the solvent of the liquid feed. Spraying the liquid feed into the cold fluid results in rapid freezing of the atomized droplets to form solid particles. The particles are collected, and then the solvent is removed, generally through sublimation (lyophilization) in a vacuum. Any known technique, such as those described by Mumenthaler et al, Int. J. Pharmaceutics (1991) 72:97-110 (1991) and Maa et al., Phar. Res., 16: 249 (1999), may be used to carry out the spray-freeze-drying step. Exemplary commercially available freeze-dryers that can be used in the spray-freeze-drying process include those manufactured by Labconco Corporation, Biolab Scientific, and AAPPTec.

For example, the spray-freeze-drying process is performed in a manner similar to spray-drying, except that instead of spraying into hot air or gas, the liquid feed is sprayed into a cold liquid or cold gas to form liquid fine droplets. An exemplary set up is depicted in FIGS. 1B and 1C. Generally, the liquid feed is atomized using known technique, for example, via a two-fluid nozzle or ultrasonic nozzle using filtered pressurized air, into the cold fluid. The cold fluid may be a liquid such as liquid nitrogen, liquid argon, or any other gas that results in the immediate freezing of the atomized droplets of the liquid feed. The cold fluid can have a temperature in a range from about −200° C. to −100° C., from about −200° C. to about −80° C., such as about −200° C. (liquid nitrogen at −196° C.). In some aspects, the cold liquid may be under stirring as the atomization process occurs. A schematic illustrating spherical porous dry powder produced by spray freeze drying is shown in FIG. 1D. A scanning electron microscopy (“SEM”) image of spherical porous dry powder produced by spray freeze drying, under 5000× magnification is shown in FIG. 1E.

The atomization conditions, including atomization air flow rate, liquid flow rate, feed rate, atomization pressure, and nozzle configuration, can be controlled as described above to produce liquid droplets having a suitable size.

The frozen droplets of the liquid feed are then freeze dried to remove frozen water, leaving particles containing the retinoid and/or retinoid derivative and the β-cyclodextrin and/or β-cyclodextrin derivative, and optionally the pharmaceutically acceptable excipient and/or additional active agent. This may be done using techniques known for lyophilization, i.e., freezing as a cake rather than as droplets. A vacuum can be applied during the second drying step. For example, the frozen droplets are freeze dried by a two-stage vacuum drying (i.e., primary drying stage and secondary drying stage) optionally under a pressure in a range from about 20 mT to about 500 mT (i.e., about 2.666 Pa to about 66.65 Pa). The primary drying stage may be performed at a temperature in a range from about −50° C. to 0° C., from about −40° C. to 0° C., or from about −40° C. to −10° C., such as −25° C., for a period from about 4 hours to about 40 hours. Frozen water is removed by ice sublimation. In the secondary drying stage, drying is normally performed at a temperature in a range from about 5° C. to 50° C., from about 10° C. to about 40° C., from about 10° C. to about 30° C., such as about 20° C. at a pressure of less than 100 mT or less than 0.15 mbar, such as from about 1 mT to about 100 mT, from about 5 mT to about 100 mT, or from about 0.001 mbar to about 0.15 mbar, from about 0.01 mbar to about 0.1 mbar, or from about 0.005 mbar to about 0.5 mbar, for a period from about 5 hours to about 24 hours. The specific spray-freeze-drying conditions used may be adjusted according to the desired properties of the particles to be produced. The resulting particles can then be collected using conventional techniques and optionally with bulking agents.

An exemplary spray-freeze-drying process uses a Labconco freeze drier with a two fluid nozzle and have the following settings: a primary drying temperature in a range from about −40° C. to −10° C., such as −25° C.; a secondary drying temperature in a range from about 10° C. to 40° C., such as 20° C.; a feed rate between about 0.1 mL/min and 30 mL/min, such as about 1.5 mL/min; an air flow rate between about 550 L/h to about 700 L/h, such as about 601 L/h; and a drying pressure between about 0.001 mbar to about 0.15 mbar, such as about 0.14 mbar, where the production yield of particles following spray drying is at least 65 wt %, such as between about 65 wt % and about 95 wt %.

### IV. Methods of Using the Dry Powder Formulations

A. Preventing, Treating, or Ameliorating Symptom(s) Associated with a Respiratory Viral Infection

Methods of using the dry powder formulation or a delivery system containing the dry powder formulation loaded in an inhaler or a nasal device for preventing, treating, or ameliorating symptoms associated with a respiratory viral infection in a subject are disclosed. The retinoid and retinoid derivatives have broad spectrum antiviral activity, and thus can be used for treating a variety of respiratory viral infections, such as those described below.

Generally, the method includes (i) administering to the subject the dry powder formulations disclosed herein. Typically, following step (i), a unit dose of the retinoid and/or retinoid derivative is delivered to the respiratory tract of the subject, such as the lung(s) and/or upper respiratory tract of the subject to prevent, treat, or ameliorate one or more symptoms associated with the respiratory viral infection in the subject.

The dry powder formulation may be administered by inhalation or intratracheal administration. For example, the dry powder formulation is administered using an inhaler, such as a dry powder inhaler, by a medical professional or the subject being treated (i.e., self-administration). In some aspects, the dry powder formulation may be administered by intranasal administration. For intranasal administration of the dry powder formulations, the powder formulation may be formulated into an aqueous solution or suspension by dissolving or suspending the dry powder formulation in a suitable solvent, such as those described above, prior to use and then administered using a nasal devices as a spray or drops, by a medical professional or the subject being treated.
1. Subject being Treated

The subject being treated using the disclosed method can be a mammal. The subject being treated typically has or at the risk of having a respiratory viral infection, such as sever acute respiratory syndrome, Middle East respiratory syndrome, Coronavirus Disease, and a flu caused by an influenza virus, and a combination thereof. For example, the subject being treated has or at the risk of getting infected by a severe acute respiratory syndrome coronavirus (e.g., SARS-CoV-2), a Middle East respiratory syndrome coronavirus (e.g., MERS-CoV), and/or an influenza virus (e.g., an influenza A virus, for example, H1N1).
2. Repeated Administrations

Typically, the administration step (i) is performed to deliver a unit dose of the retinoid and/or retinoid derivative to the respiratory tract of the subject. The administration step (i) may be repeated to deliver multiple unit doses of the retinoid and/or retinoid derivative to the subject.

The administration step (i) may be repeated at least one time, at least two times, at least three times, at least five times, at least ten times, at least twenty times, up to thirty times, or more than thirty times. For example, the administration step (i) is repeated one time, two times, three times, five times, ten times, fifteen times, twenty times, or thirty times.

The period for repeated administration of the dry powder formulation can be between one day and 6 months, between one day and 3 months, between one and thirty days, between one and ten days, between one and three days, between one and two days, or during one day.

For example, the administration step (i) is repeated one time, two times, three times, five times, ten times, fifteen times, twenty times, or thirty times or more for a period between one day and 6 months, between one day and 3 months, between one and thirty days, between one and ten days, between one and three days, between one and two days, or during one day.

The administration step (i) may be repeated consecutively following the previous administration. For example, the administration is repeated within 10 minutes, within 8 minutes, within 5 minutes, within 3 minutes, within 2 minutes, within 1 minute, or within 30 seconds following the previous administration.

Optionally, the administration step is repeated regularly at a different time. For example, the administration may be performed at a frequency, such as every hour, every 2 hours, every 5 hours, every 8 hours, every day, every 2 days, every 3 days, every 5 days, every 7 days, every 10 days, every two weeks, or every month. For example, the administration step (i) is repeated every hour, every 2 hours, every 5 hours, every 8 hours, every day, every 2 days, every 3 days, every 5 days, every 7 days, every 10 days, every two weeks, or every month for a period between one day and 6 months, between one day and 3 months, between one and thirty days, between one and ten days, between one and three days, between one and two days, or during one day.

Alternatively, the administration may be repeated irregularly, for example, repeating the administration 1 day after the first administration, then 2 days after the second administration, then 5 days after the third administration, then 7 day after the fourth administration, and then 30 days after the fifth administration. The time interval between administrations are determined based on the patient's needs.
3. Effective Amounts

Typically, following a single administration step or all of the administration steps, an effective amount of the retinoid and/or retinoid derivative is delivered to the respiratory tract, such as the lower and/or upper respiratory tract(s), of the subject to prevent, treat, or ameliorate symptoms associated with any one or more of the above-described respiratory viral infections in the subject.

In some aspects, following a single administration step or all of the administration steps, the effective amount of the retinoid and/or retinoid derivative delivered to the respiratory tract of the subject is effective to show prophylactic protection of the subject against a variety of viral infections, such as those described above.

For example, the subject is infected by a severe acute respiratory syndrome coronavirus (e.g., SARS-CoV-2), a Middle East respiratory syndrome coronavirus (e.g., MERS-CoV), and/or an influenza virus (e.g., an influenza A virus, for example, H1N1) after the single administration step or all of the administration steps, and the effective amount of retinoid and/or retinoid derivative delivered to the respiratory tract of the subject is effective to reduce the viral load in the lung of the subject compared to the viral load in the lung of a control, to reduce the degree of lung damage compared to the degree of lung damage in the control, and/or to reduce the expression of a viral protein compared to the expression of the viral protein in the control. The control is the same species as the subject that is administered with an unformulated retinoid and/or retinoid derivative or a vehicle control, such as a buffer, by the same administration route and is infected by the same virus as the subject.

For example, the subject is infected by a severe acute respiratory syndrome coronavirus (e.g., SARS-CoV-2), a Middle East respiratory syndrome coronavirus (e.g., MERS-CoV), and/or an influenza virus (e.g., an influenza A virus, for example, H1N1) after the single administration step or all of the administration steps, and the effective amount of retinoid and/or retinoid derivative delivered to the respiratory tract of the subject is effective to reduce the viral load in the lung of the subject by at least 50% compared to the viral load in the lung of a control, to reduce the degree of lung damage, such as reduced degree of bronchiolar and alveolar cell infiltrations as shown by histopathological morphology, compared to the degree of lung damage in the control, and/or to reduce the expression level of a viral protein in the lung of the subject, such as a SARS-CoV-2 nucleocapsid protein and/or an influenza virus PA protein, by at least 20%, compared to the expression of the viral protein in the lung of the control. Methods for measuring the viral load and expression levels of viral proteins in the lung are known, for example, using qRT-PCR, plaque assay, and western blot as described in Yuan, et al., Nature Communications 2019, 10, 120.

In some aspects, following a single administration step or all of the administration steps, the effective amount of the retinoid and/or retinoid derivative delivered to the respiratory tract of the subject is effective to show therapeutic effects in a subject having a viral infection, such as any one or more of the viral infections described above.

For example, the subject is infected by a severe acute respiratory syndrome coronavirus (e.g., SARS-CoV-2), a Middle East respiratory syndrome coronavirus (e.g., MERS-CoV), and/or an influenza virus (e.g., an influenza A virus, for example, H1N1) prior to the single administration step or all of the administration steps, and the effective amount of retinoid and/or retinoid derivative delivered to the respiratory tract of the subject is effective to increase the survival rate and/or to reduce the viral load in the lung of the subject, compared to the survival rate and/or viral load in the lung of a control. The control is the same species as the subject that is administered with an unformulated retinoid and/or retinoid derivative or a vehicle control, such as a buffer, by the same administration route and is infected by the same virus as the subject. The term “survival rate” refers to the chance of survival of a subject by the end of a defined time period following infection by a virus.

For example, the subject is infected by a severe acute respiratory syndrome coronavirus (e.g., SARS-CoV-2), a Middle East respiratory syndrome coronavirus (e.g., MERS-CoV), and/or an influenza virus (e.g., an influenza A virus, for example, H1N1) prior to the single administration step or all of the administration steps, and the effective amount of retinoid and/or retinoid derivative delivered to the respiratory tract of the subject is effective to increase the survival rate by at least 50% compared to the survival rate of the control, and/or to reduce the viral load in the lung of the subject by at least 50% compared to the viral load in the lung of the control.

In some aspects, following a single administration step or all of the administration steps, the effective amount of the retinoid and/or retinoid derivative delivered to the respiratory tract of the subject is effective to show prophylactic protection of the subject against a viral infection, such as severe acute respiratory syndrome, Middle East respiratory syndrome, and/or flu caused by an influenza virus, that is comparable to or better than a commercially available treatment option, such as remdesivir and zanamivir.

For example, the subject is infected by a severe acute respiratory syndrome coronavirus, such as SARS-CoV-2, after the single administration step or all of the administration steps, and the effective amount of retinoid and/or retinoid derivative delivered to the respiratory tract of the subject is effective to reach a viral load, a degree of lung damage, and/or an expression level of viral protein in the lung of the subject that are/is comparable to the viral load, degree of lung damage, and/or expression level of viral protein in the lung of a control that is administered with remdesivir at the same dose as the retinoid and/or retinoid derivative and then infected by SARS-CoV-2, both performed at the same time point as the subject. “Comparable” means that the viral load and expression level of the viral protein in the lung measured by qRT-PCR is within ±10%, and the degree of bronchiolar and alveolar cell infiltrations as shown by histopathological morphology are no observable differences.

For example, the subject is infected by an influenza A virus, such as H1N1, after the single administration step or all of the administration steps, and the effective amount of retinoid and/or retinoid derivative delivered to the respiratory tract of the subject is effective to reduce the viral load in the lung of the subject by at least 50% compared to the viral load in the lung of a control administered with zanamivir at the same dose as the retinoid and/or retinoid derivative and infected by H1N1, both performed at the same time point as the subject.

In some aspects, following a single administration step or all of the administration steps, the effective amount of the retinoid and/or retinoid derivative delivered to the respiratory tract of the subject is effective to show therapeutic effects in a subject having a viral infection, such as severe acute respiratory syndrome, Middle East respiratory syndrome, and/or flu caused by an influenza virus, that is comparable to or better than a commercially available treatment option, such as remdesivir and zanamivir.

For example, the subject is infected by an influenza A virus, such as H1N1, prior to the single administration step or all of the administration steps, and the effective amount of retinoid and/or retinoid derivative delivered to the respiratory tract of the subject is effective to increase the survival rate of the subject by at least 20% compared to the survival rate of a subject administered with zanamivir at the same dose as or a lower dose than the retinoid and/or retinoid derivative and infected by H1N1, and/or to reduce the viral load in the lung of the subject by at least 50% compared to the viral load in the lung of a control administered with zanamivir at the same dose as or a lower dose than the retinoid and/or retinoid derivative and infected by H1N1, both performed at the same time point as the subject.

In some aspects, during each administration step (i), the dosage of the retinoid and/or retinoid derivative in the dry powder formulation or the solution or suspension formed by the dry powder formulation can be in a range from about 1 mg to about 3000 mg, from about 1 mg to about 1500 mg, from about 10 mg to about 1500 mg, from about 10 mg to about 1000 mg, from about 20 mg to about 1000 mg, from about 50 mg to about 1500 mg, from about 50 mg to about 1000 mg, or from about 20 mg to about 500 mg.

In some aspects, during each administration step (i), the dosage of the retinoid and/or retinoid derivative in the dry powder formulation or the solution or suspension formed by the dry powder formulation can be in a range from about 0.1 mg to about 100 mg, from about 0.1 mg to about 50 mg, from about 0.1 mg to about 10 mg, from about 0.1 mg to about 5 mg, from about 0.5 mg to about 100 mg, from about 0.5 μg to about 50 mg, from about 0.5 mg to about 20 mg, from about 0.5 mg to about 10 mg, from about 1 mg to about 100 mg, from about 1 mg to about 50 mg, from about 1 μg to about 20 mg, from about 1 mg to about 10 mg, from about 5 mg to about 100 mg, or from about 5 mg to about 50 mg per kg of the subject being treated.
4. Optional Steps

In addition to step (i) administering the dry powder formulation to the subject described above, the method may include a step of loading the dry powder formulation into the inhaler prior to step (i).

In some aspects, a user, such as a medical professional or the subject being treated, can load the dry powder formulation into the reservoir of an inhaler for delivering the dry powder formulation to the subject. The dry powder formulation loaded in the reservoir of the inhaler may contain one unit dose of the retinoid and/or retinoid derivative (i.e., dry powder formulation in unit dosage form) or multiple unit doses of the retinoid and/or retinoid derivative. When the dry formulation containing multiple unit doses of the retinoid and/or retinoid derivative is loaded in the reservoir of the inhaler, a metered valve is typically included in the inhaler such that each administration delivers one unit dose of the retinoid and/or retinoid derivative.

Optionally, a capsule or replaceable set prepackaged with the dry powder formulation is provided. Typically, the dry powder formulation prepackaged in the capsule or replaceable set is in a unit dosage form, i.e., contain a unit dose of the retinoid and/or retinoid derivative. For example, the capsule is prepackaged with one dry powder formulation in unit dosage form. The user can load the one prepackaged capsule or two or more of the prepackaged capsules in the inhaler.

For example, the replaceable set is a foil-foil blister prepackaged with several unit doses of the dry powder formulations in unit dosage form, where each unit dose is spatially separated from the other unit doses (i.e., discrete unit doses). The user can load the prepackaged foil-foil blister in the inhaler and optionally replace with another prepackaged foil-foil blister after all doses are delivered to the subject.

In some aspects, the dry powder formulation is administered intranasally as drops or a spray. In these cases, the user can prepare a solution or suspension by dissolving or suspending the dry powder formulation in a suitable solvent, such as distilled water, de-ionized water, pure or ultrapure water, saline, phosphate-buffered saline (PBS), Ringer's solution, and isotonic sodium chloride, and a combination thereof, and then load the solution or suspension formed by the dry powder formulation into the reservoir of a nasal device for delivering the dry powder formulation to the subject. Optionally, a container prepackaged with a solution or suspension formed by the dry powder formulations is provided to the user.

The dry powder formulation forming the solution or suspension may contain one unit dose of the retinoid and/or retinoid derivative (i.e., dry powder formulation in unit dosage form) or multiple unit doses of the retinoid and/or retinoid derivative. When the solution or suspension formed by dry formulations containing multiple unit doses of the retinoid and/or retinoid derivative is loaded in the reservoir of the nasal device, a metered valve can be included in the device such that each administration delivers one unit dose of the retinoid and/or retinoid derivative.

The disclosed compositions and methods can be further understood through the following numbered paragraphs.

1. A dry powder formulation for inhalation or intratracheal administration and/or for intranasal administration comprising particles comprising
a retinoid or a retinoid derivative, or a combination thereof; and
a β-cyclodextrin or a β-cyclodextrin derivative, or a combination thereof,
wherein the amount of the β-cyclodextrin or the β-cyclodextrin derivative, or the total amount of the β-cyclodextrin and β-cyclodextrin derivative is at least 20 wt % of the total amount of the retinoid and/or retinoid derivative and the β-cyclodextrin and/or β-cyclodextrin derivative.
   2. The dry powder formulation of paragraph 1, wherein the particles are porous and spherical in shape.
   3. The dry powder formulation of paragraph 1 or paragraph 2, wherein the retinoid and/or retinoid derivative form a complex with the β-cyclodextrin and/or β-cyclodextrin derivative via hydrophobic interactions.
   4. The dry powder formulation of any one of paragraphs 1-3, wherein the retinoid and/or retinoid derivative are/is in an amorphous form.
   5. The dry powder formulation of any one of paragraphs 1-4, wherein the particles comprise retinol, tretinoin, isotretinoin, alitretinoin, etretinate, acitretin, adapalene, bexarotene, tazarotene, or tamibarotene, or a combination thereof.
   6. The dry powder formulation of any one of paragraphs 1-5, wherein the particles comprise a β-cyclodextrin derivative, and wherein the β-cyclodextrin derivative is 2-hydroxypropyl-β-cyclodextrin, methyl-β-cyclodextrin, or sulfobutylether β-cyclodextrin, or a combination thereof.
   7. The dry powder formulation of any one of paragraphs 1-6, wherein particles comprise tamibarotene and 2-hydroxypropyl-β-cyclodextrin.
   8. The dry powder formulation of any one of paragraphs 1-7 further comprising a pharmaceutically acceptable excipient, an additional active agent, or a combination thereof.
   9. The dry powder formulation of paragraph 8, wherein the pharmaceutically acceptable excipient is an amino acid, a peptide, a lipid, a protein, a chelating agent, a salt, a taste masking agent, a cation, or a polymer, or a combination thereof.
   10. The dry powder formulation of paragraph 8 or paragraph 9, wherein the amount of the pharmaceutically acceptable excipient is in a range from 0.1 wt % to 20 wt %, from, from 0.1 wt % to 15 wt %, from 1 wt % to 12 wt %, from 1 wt % to 10 wt %, from 1 wt % and 15 wt %, from 2 wt % to 20 wt %, from 2 wt % to 15 wt %, from 2 wt % to 10 wt %, from 3 wt % to 20 wt %, from 3 wt % to 15 wt %, or from 3 wt % to 10 wt % of the dry powder formulation.
   11. The dry powder formulation of any one of paragraphs 8-10, wherein the additional active agent is an anti-viral agent or anti-inflammatory agent, or a combination thereof.
   12. The dry powder formulation of any one of paragraphs 1-11, wherein the dry powder formulation is for inhalation, and wherein the particles have a mass median aerodynamic diameter (“MMAD”) of <5 μm, <4 μm, <3.5 μm, <3 μm, <2.5 μm, or <2 μm.
   13. The dry powder formulation of paragraph 12, wherein the particles have a volumetric mean diameter that is larger than the MMAD of the particles, and wherein the volumetric mean diameter of the particles is >4 μm, >5 μm, >8 μm, >10 μm, >12 μm, >15 μm, in a range from 4 μm to 20 μm, from 4 μm to 15 μm, or from 4 μm to 15 μm.
   14. The dry powder formulation of paragraph 12 or paragraph 13, wherein the particles have a fine particle fraction>40%, >45%, >50%, >55%, >60%, or ≥65% in cascade impactor study.
   15. The dry powder formulation of any one of paragraphs 1-11, wherein the dry powder formulation is for intranasal administration, and wherein the particles have a mass median aerodynamic diameter>9 μm, >9.5 μm, >10 μm, or >10.5 μm.
   16. The dry powder formulation of paragraph 15, wherein the particles have a volumetric mean diameter of >50 μm, >55 μm, >60 μm, >65 μm, in a range from 50 μm to 80 μm, from 50 μm to 75 μm, or from 50 μm to 70 μm.
   17. The dry powder formulation of paragraph 15 or paragraph 16, wherein the particles have a fraction of particles>9 μm of >40%, >45%, >50%, >55%, or >60% in Andersen Cascade Impactor (“ACI”) study.
   18. A delivery system comprising an inhaler and the dry powder formulation of any one of paragraphs 1-14 in a unit dosage form.
   19. The delivery system of paragraph 18, wherein the inhaler is a dry powder inhaler or a pressurized metered dose inhaler.
   20. The delivery system of paragraph 18 or paragraph 19, wherein the total amount of the retinoid and/or retinoid derivative in the unit dosage form is in a range from about 0.1 mg to about 50 mg, from about 0.5 to about 50 mg, or from about 1 to about 50 mg.
   21. The delivery system of any one of paragraphs 18-20, wherein the emitted fraction of the particles is >65%, >70%, >75%, >80%, >85%, >90%, >92%, or >95%.
   22. A delivery system comprising a nasal device and a solution or suspension formed by the dry powder formulation of any one of paragraphs 1-11 and 15-17 in a unit dosage form and a solvent, wherein the particles of the dry powder formulation are dissolved or suspended in the solvent.
   23. The delivery system of paragraph 22, wherein the nasal device is a metered dose spray pump, an atomiser, a syringe, a bulb, a canister, a pressurized container, a spray can, or a nebulizer.
   24. The delivery system of paragraph 22 or paragraph 23, wherein the total amount of the retinoid and/or retinoid derivative in the unit dosage form is in a range from about 0.1 mg to about 50 mg, from about 0.5 to about 50 mg, or from about 1 to about 50 mg.
   25. The delivery system of any one of paragraphs 22-24, wherein the emitted fraction of the particles is >85%, >90%, or >95%.
   26. A method of making the dry powder formulation of any one of paragraphs 1-17 comprising
(i) mixing a retinoid and/or a retinoid derivative and a β-cyclodextrin and/or a β-cyclodextrin derivative, and optionally a pharmaceutically acceptable excipient and/or an additional active agent, in a solvent to form a liquid feed; and
(ii) spray-drying or spray-freeze drying the liquid feed to form particles containing the retinoid and/or a retinoid derivative and the β-cyclodextrin and/or β-cyclodextrin derivative, and optionally the pharmaceutically acceptable excipient and/or the additional active agent.
   27. The method of paragraph 26, wherein following step (ii), the production yield of the particles is at least 40 wt %, at least 50 wt %, at least 60 wt %, at least 65 wt %, at least 70 wt %, at least 75 wt %, at least 80 wt %, at least 85 wt %, at least 90 wt %, in a range from about 40 wt % to about 95 wt %, from about 55 wt % to about 95 wt %, from about 55 wt % to about 95 wt %, from about 60 wt % to about 95 wt %, or from about 65 wt % to about 95 wt %.
   28. The method of paragraph 26 or paragraph 27, wherein during step (ii), the liquid feed is sprayed through a two-fluid nozzle.
   29. A method of preventing, treating, or ameliorating symptom(s) associated with a respiratory viral infection in a subject, the method comprising (i) administering to the subject the dry powder formulation of any one of paragraphs 1-17.
   30. The method of paragraph 29, wherein the subject is a mammal having or at the risk of having sever acute respiratory syndrome, Middle East respiratory syndrome, Coronavirus Disease, or a flu caused by an influenza virus, or a combination thereof.
   31. The method of paragraph 29 or paragraph 30, wherein in step (i), the dry powder formulation is administered by inhalation or intratracheal administration.
   32. The method of paragraph 29 or paragraph 30, wherein prior to step (i), the dry powder formulation is mixed with a solvent to form a solution or suspension, and wherein in step (i), the solution or suspension is administered by intranasal administration.
   33. The method of any one of paragraphs 29-32 further comprising repeating step (i) every hour, every 2 hours, every 5 hours, every 8 hours, every day, every 2 days, every 3 days, every 5 days, every 7 days, every 10 days, every two weeks, or every month.
   34. The method of paragraph 33, wherein step (i) is repeated for a period between one day and 6 months, between one day and 3 months, between one and thirty days, between one and ten days, between one and three days, or during one day.
   35. The method of any one of paragraphs 29-34, wherein following a single administration step or all of the administration steps, an effective amount of the retinoid and/or retinoid derivative is delivered to the lower and/or upper respiratory tract of the subject.
   36. The method of paragraph 35, wherein the subject is infected by a severe acute respiratory syndrome coronavirus, a Middle East respiratory syndrome coronavirus, and/or an influenza virus after the single administration step or all of the administration steps, and wherein the effective amount of retinoid and/or retinoid derivative is effective to reduce the viral load in the lung of the subject compared to the viral load in the lung of a control, to reduce the degree of lung damage compared to the degree of lung damage in the control, and/or to reduce the expression level of a viral protein compared to the expression of the viral protein in the control.
   37. The method of paragraph 35, wherein the subject is infected by a severe acute respiratory syndrome coronavirus, a Middle East respiratory syndrome coronavirus, and/or an influenza virus prior to the single administration step or all of the administration steps, and wherein the effective amount of retinoid and/or retinoid derivative is effective to increase the survival rate and/or to reduce the viral load in the lung of the subject, compared to the survival rate and/or viral load in the lung of a control.
   38. The method of any one of paragraphs 29-37, wherein during step (i), the dosage of the retinoid and/or retinoid derivative in the dry powder formulation or the solution or suspension formed from the dry powder formulation administered is in a range from about 0.1 mg to about 100 mg, from about 0.1 mg to about 50 mg, from about 0.1 mg to about 10 mg, from about 0.1 mg to about 5 mg, from about 0.5 mg to about 100 mg, from about 0.5 μg to about 50 mg, from about 0.5 mg to about 20 mg, from about 0.5 mg to about 10 mg, from about 1 mg to about 100 mg, from about 1 mg to about 50 mg, from about 1 μg to about 20 mg, from about 1 mg to about 10 mg, from about 5 mg to about 100 mg, or from about 5 mg to about 50 mg per kg of the subject.

The present invention will be further understood by reference to the following non-limiting examples.

### EXAMPLES

### Example 1. Dry Powder Formulations of Tamibarotene Show Properties for Pulmonary and/or Nasal Delivery

Materials and Methods

Materials

Tamibarotene was purchased from Cayman Chemical (Michigan, USA). 2-hydroxypropyl-β-cyclodextrin (HPβCD) was purchased from Sigma-Aldrich (Saint Louis, USA). Tert-butyl alcohol (TBA) was obtained from Meryer Chemical Technology (Shanghai, China). Methanol and acetonitrile (HPLC grade) were purchased from Anaqua Chemicals Supply (Cleveland, USA). Acetic acid (HPLC grade) was obtained from Fisher Scientific (Loughborough, UK). All solvents and reagents were of analytical grade or better unless otherwise stated.

Spray Freeze Drying of Tamibarotene

The feed solution for SFD was first prepared by mixing the stock solutions of tamibarotene (10 mg/mL in TBA) and HPβCD (100 mg/mL in water) at 1:2 tamibarotene: HPβCD molar ratio to a final total solute concentration of 52.1 mg/mL. The solution was mixed and maintained at 37° C. (to prevent the freezing of TBA which has a freezing point of 25.4° C.) prior to spraying. A two-fluid nozzle (Büchi, stainless steel two-fluid nozzle with an internal diameter of 0.7 mm, Switzerland) operated at a gas flow rate of 601 L/h was used for atomization. The feed solutions were loaded into a syringe which was connected to the nozzle and the liquid feed rate was controlled at 1.5 mL/min by a syringe pump (LEGATO® 210 Syringe Pump, KD Scientific, MA, USA). The atomized droplets were collected in a stainless-steel collector containing liquid nitrogen to allow instant freezing. The frozen droplets were transferred into a freeze dryer (FreeZone® 6 Liter Benchtop Freeze Dry System with Stoppering Tray Dryer, Labconco Corporation, Missouri, USA) which was programmed to maintain a primary drying temperature at −25° C. for 40 h, followed by a secondary drying in which the temperature was gradually increased to 20° C. in 4 h and the temperature was maintained for at least 20 h. The samples were kept under a pressure below 0.14 mBar throughout the freeze-drying process. The dried products were collected and stored in desiccators with silica gel at ambient temperature until further analysis. The production yield was calculated as the percentage of the total mass of powder collected in the initial solute mass input. A schematic showing the two steps involved in spray freeze drying: (i) spray freezing, atomization of liquid by a nozzle into cryogen forming frozen particles; and (ii) freeze drying, sublimation of solvent and formation of dried porous particles, is depicted in FIGS. 1B and 1C. FIGS. 1D and 1E show the schematic and scanning electron microscopy image of spherical porous dry powder produced by spray freeze drying, respectively.

Drug Quantification by High Performance Liquid Chromatography

Tamibarotene was quantified using high performance liquid chromatography (HPLC) with photodiode array detector (Agilent 1260 Infinity; Santa Clara, USA). A C-18 column (Agilent Prep—C18, 4.6×250 mm, 5 μm) was used with a mobile phase composed of acetonitrile and 5% acetic acid 80/20 (v/v). A volume of 25 μL was injected and the running flow rate was set at 1 mL/min. Tamibarotene was detected at 280 nm with a retention time at 6.2 min. Tamibarotene was quantified against a standard curve in the range of 1.57 to 200 μg/mL. To determine the drug loading, SFD powder of tamibarotene were weighed and dissolved in methanol to a final volume of 5 mL. The sample was filtered through a 0.45-μm nylon membrane filter before quantified by HPLC as described above. The measurement of drug content in each formulation was performed in triplicate. Drug loading is defined as the ratio of tamibarotene detected in the formulation to the total amount of powder.

Scanning Electron Microscopy (SEM)

The morphology of A2-TFN powder and the unformulated drug was visualized by field emission SEM (Hitachi S-4800 N, Tokyo, Japan) at 5 kV. The powders were sprinkled onto carbon stick tape which was mounted on SEM stubs, and excess powders were removed by clean air. The powders were sputter-coated using a sputter coater (Q150T PLUS Turbomolecular Pumped Coater, Quorum, UK) with approximately 13 nm gold-palladium alloy in 90 s to avoid charging during SEM imaging.

Evaluation of Aerosol Performance by Cascade Impactors

The aerosol performance of A2-TFN powder for pulmonary delivery was evaluated using a Next Generation Impactor (NGI, Copley, Nottingham, UK) as previously described. Briefly, approximately 3 mg of A2-TFN powders were weighed and loaded into a size 3 capsules which were placed in a Breezhaler®. The operating airflow rate was set at 90 L/min with a pressure drop of 3.4 kPa. Prior to each dispersion, a thin layer of silicon grease (LPS Laboratories, Illinois, GA, USA) was coated onto the stages of NGI to reduce particle bounce. After dispersion of two capsules, methanol was used to rinse and dissolve the powder deposited on capsule, inhaler, adaptor and each stage of NGI. The recovered dose was defined as the total mass of tamibarotene assayed by HPLC on all stages in a single run of impaction. The emitted fraction (EF) referred to the fraction of powder that exited the inhaler with respect to the recovered dose. Fine particle dose (FPD) was the mass of particles with aerodynamic diameter less than 5.0 μm as calculated with the assayed tamibarotene obtained from HPLC in NGI experiment. Fine particle fraction (FPF) was defined as the percentage fraction of FPD with respect to the recovered dose.

Dissolution Study

The dissolution profile of A2-TFN powders was investigated using a jacketed beaker which contained 100 mL of simulated lung fluid as dissolution medium. The preparation of simulated lung fluid was according to Marques et al. (SLF3 in the article). The temperature was maintained at 37° C. and the medium was stirred at 75 rpm with a magnetic bar. As fine particle dose (FPD, aerodynamic diameter<5 μm) is considered to be the fraction of powder that can deposit in the deep lung region, the FPD of A2-TFN formulation was collected by a Fast Screening Impactor (FSI, Copley Scientific, UK) coupled with Breezhaler® as described before for dissolution study. Approximately 8.5±0.5 mg of A2-TFN powder was dispersed by FSI to separate an FPD (which contained an estimated amount of 0.5 mg of tamibarotene). The powders were placed on a glass fiber filter paper which was then transferred into the jacketed beaker. At pre-determined time intervals, 1 mL of dissolution medium was withdrawn and filtered through 0.45-μm membrane filter. Equal volume of pre-warmed fresh medium was refilled immediately. The unformulated tamibarotene powder was included as control for comparison. The concentration of tamibarotene was quantified by HPLC as described above. The dissolution study was carried out in triplicate.

Fourier-Transform Infrared (FT-IR) Spectroscopy

Fourier transform infrared spectroscopy (FT-IR) spectrum of A2-TFN powders and raw materials were obtained using Spectrum Two FT-IR spectrometer with UATR Accessory (PerkinElmer, USA). Appropriate amount of powder (less than 1 mg) was placed on the surface of the UATR crystal and pressed to form pellets. For each sample, the scanning range of spectra was 400-4000 cm⁻¹.

Differential Scanning Calorimetry (DSC)

Differential scanning calorimetry (DSC) (DSC 250, TA Instruments, Newcastle, Del., USA) was used to study the thermal response of raw materials and their physical mixture, as well as A2-TFN powders. Approximately 1 mg of powders were weighed and loaded into an aluminum crucible and heated from 50° C. to 280° C. at a constant rate of 10° C./min.

Results

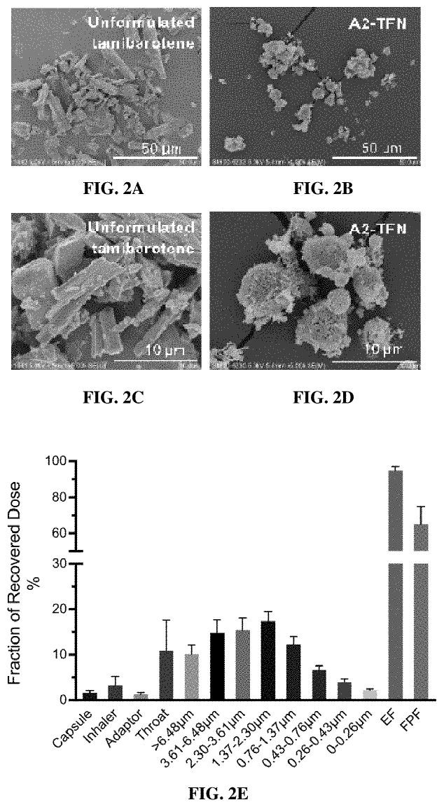

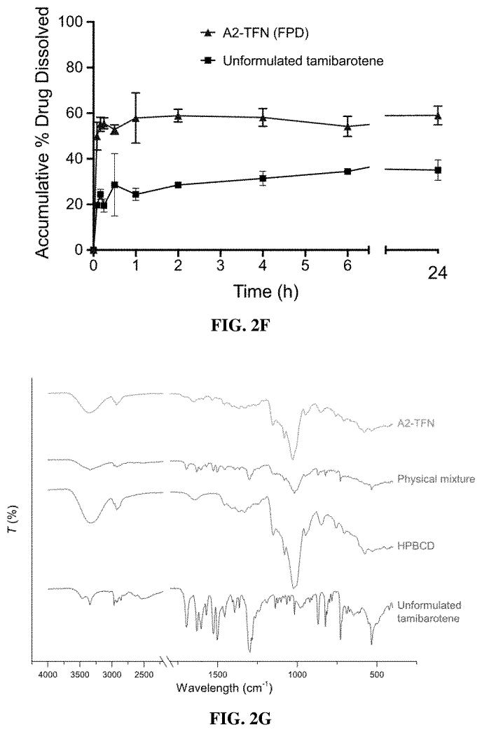

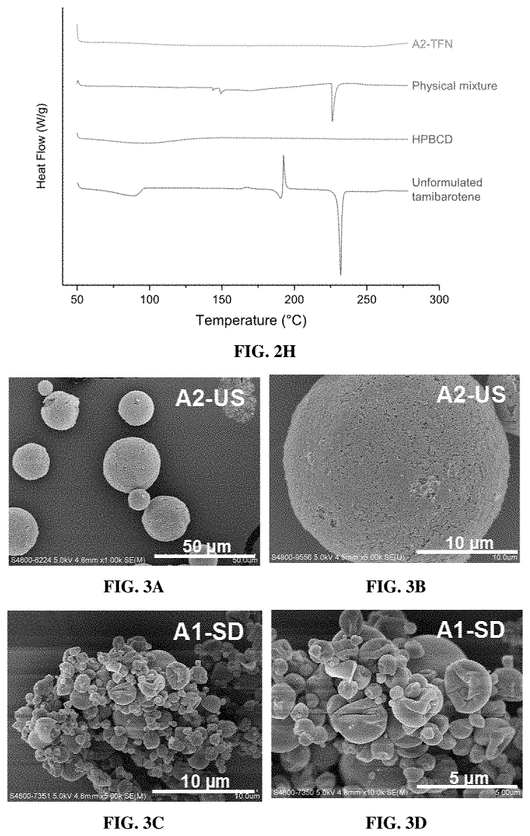

Three dry powder formulations of tamibarotene have been prepared with different drying conditions (Table 1). The formulations show favorable aerosol properties for inhalation. In particular, A2-TFN formulation, which contains HPβCD and tamibarotene at 2:1 molar ratio prepared by SFD using a two-fluid nozzle for atomization, has been used for further investigation. The production yield of A2-TFN is 66.3% and the measured drug loading is closed to the theoretical value of 10.2% w/w. The morphology of tamibarotene powder before and after SFD has been visualized by scanning electron microscopy (SEM) (FIGS. 2A-2D). Compared to the unformulated tamibarotene which displays rod-shape crystalline structure with varied length, A2-TFN powder shows porous and spherical structures. Particles of A2-TFN formulation are small in size (<10 μm) and the particles appear to be slightly aggregated. The volumetric particle size distribution measured with laser diffractometer is consistent with SEM images, with a median diameter of 5.75±0.11 μm (Table 1). For inhaled formulation, aerodynamic diameter can determine the site of lung deposition following powder dispersion. Aerodynamic diameter between 1 to 5 μm is considered to be acceptable for lung deposition. The aerodynamic diameter of A2-TFN formulation has been measured by Next Generation Impactor (NGI) coupled with a Breezhaler® operated at 90 L/min (FIG. 2E). The mass median aerodynamic diameter (MMAD) has been calculated as 1.86±0.44 μm, which is within the particle size range for effective lung deposition. The emitted fraction (EF) and fine particle fraction (FPF) are around 95% and 65%, respectively, demonstrating that A2-TFN powder can exit the inhaler and aerosolize efficiently with excellent lung deposition. The MMAD of the SFD powder is smaller than its volumetric size. This can be attributed to the low density of porous particles which facilitate powder aerosolization and reduce interparticle attraction. Moreover, particles with small aerodynamic diameter but large geometric size have the additional advantage of efficient lung deposition yet prolonged retention in the airway by avoiding rapid clearance. The aerosol performance of the powder can be also affected by the choice of inhaler device for powder dispersion. The results show that Breezhaler® is a compatible inhaler device to the powder formulation for efficient aerosolization. The results from physicochemical and aerosol characterization demonstrate that A2-TFN formulation allows efficient powder deposition at the lower airways which coincides with the primary site of respiratory infection.

A good aqueous solubility is needed so drugs can be absorbed at concentrations needed for robust antiviral effect. As tamibarotene has a poor solubility, its solubility and dissolution rate need to be improved. Dissolution study has been performed with the fine particle dose (which reflected the dose deposited in the lower airways) of the A2-TFN formulation. A burst-release profile has been observed with a faster dissolution rate than the unformulated tamibarotene (FIG. 2F). In A2-TFN formulation, approximately 50% of tamibarotene is released in the medium within the first 5 min and the cumulative drug concentration remains steady in the next 4 h. At the end of the experiment (24 h), approximately 60% of drug has been dissolved. There may be recrystallization of tamibarotene due to supersaturation when tamibarotene is released from drug-cyclodextrin complex at the initial phase of the dissolution. In contrast, the unformulated tamibarotene dissolves in a slow and steady pace, with approximately 20% of drug dissolved within the first hour. After 24 h, the cumulative dissolved drug from unformulated tamibarotene is significantly lower than that from A2-TFN powder (Student's t-test, p<0.01), showing that HPβCD in the complex can improve the solubility of tamibarotene in the dissolution medium.

The complexation between HPβCD and tamibarotene has been reflected by the Fourier-transform infrared (FT-IR) spectrum (FIG. 2G). Characteristic peaks of tamibarotene (823, 1630, 1695, 3038 cm⁻¹) can be observed in the spectrum of unformulated tamibarotene and physical mixture, but absent in the spectrum of A2-TFN, showing the successful formation of complex between the two components, thereby facilitating the formation of hydrogen bond with water. In the differential scanning calorimetry (DSC) thermogram (FIG. 2H), the endothermic peak at 231° C. corresponding to the melting point of tamibarotene has been observed in unformulated tamibarotene and physical mixture, demonstrating that tamibarotene is in crystalline form before spray freeze drying. HPβCD and A2-TFN powder is amorphous as no significant thermal event has been recorded. The transformation of tamibarotene from crystalline form to amorphous form after SFD also contributes to a faster dissolution rate. The enhanced dissolution rate can lead to fast drug absorption before the undissolved powders are removed from the airways by the phagocytosis clearance action.

Inhalable tamibarotene powder by other drying methods (e.g., spray drying) has also been prepared and formulated as nasal powder to facilitate antiviral action in the upper airways (Table 1 and FIGS. 3A-3D).

TABLE 1
Physicochemical characterizations and aerosol performances of tamibarotene powders prepared by different drying methods.

| | Drying | | | | | | | |
|---|---|---|---|---|---|---|---|---|
| | method | Intended | | Drug | | | | |
| | (Type of | route of | Production | loading | Volumetric diameter (μm) | | | Aerosol |
| Formulation | nozzle) | administration | yield (%) | (% w/w) | D₁₀ | D₅₀ | D₉₀ | performance |
| A2-TFN | Spray freeze | Inhalation | 66.3 | 10.1 ± 0.39 | 2.27 ± 0.02 | 5.75 ± 0.11 | 14.11 ± 0.57 | EF: 94.8 ± 0.32% |
| | drying | | | | | | | FPF: 65.0 ± 0.50% |
| | (Two-fluid | | | | | | | MMAD: 1.86 ± 0.44 μm |
| | nozzle) | | | | | | | |
| A2-US | Spray freeze | Intranasal | 93.9 | 10.0 ± 0.25 | 5.22 ± 0.71 | 32.03 ± 2.79 | 66.58 ± 2.77 | EF: 95.4 ± 1.8% |
| | drying | | | | | | | FP9: 62.8 ± 2.9% |
| | (Ultrasonic | | | | | | | |
| | nozzle) | | | | | | | |
| A1-SD | Spray drying | Inhalation | 40.5 | 19.7 ± 0.61 | 0.74 ± 0.01 | 1.85 ± 0.01 | 4.37 ± 0.13 | EF: 70.4 ± 0.77% |
| | (Two-fluid | | | | | | | FPF: 49.7 ± 3.69% |
| | nozzle) | | | | | | | MMAD: 4.73 ± 0.57 μm |

The volumetric particle size distribution was measured by laser diffractometer. During measurement, A2-TFN and SD-A powders was dispersed by Breezhaler ® operated at 60 L/min, while A2-US powder was dispersed by Aptar Unidose system (UDS) for intranasal administration. Aerosol performance of inhalation formulation was evaluated by Next Generation Impactor coupled with a Breezhaler ® operated at 90 L/min. Aerosol performance of intranasal formulation was evaluated by reduced Andersen Cascade Impactor and the powder was dispersed by a Aptar UDS nasal device. The volumetric particle size was presented as D₁₀, D₅₀, and D₉₀, which represent the equivalent spherical volume diameters at 10%, 50% and 90% cumulative volume, respectively. The emitted fraction (EF) referred to the fraction of powder that exited the nasal device or inhaler with respect to the recovered dose. Fine particle fraction (FPF) was defined as the percentage fraction of particle with aerodynamic diameter less than 5 μm with respect to the recovered dose. MMAD refers to mass median aerodynamic diameter. Fraction of particles >9 μm (FP9) was the mass of particles with aerodynamic diameter over 9.0 μm in ACI experiment. Data for drug content and volumetric diameter were presented as mean ± standard deviation (n = 3).

### Example 2. Pulmonary Delivery of Powder Formulations of Tamibarotene Shows Enhanced Drug Absorption and Bioavailability

Materials and Methods

The mice were randomly allocated to two treatment groups with 45 mice per group. The first group received 1 mg of A2-TFN powder formulation through i.t. administration under anesthesia. The powder aerosolization after i.t. insufflation was evidenced by in vivo fluorescence imaging. In vivo biodistribution of A2-TFN-fluorescein powder at 0.5 and 1 hour after intratracheal administration was measured as follows: one milligram of A2-TFN-fluorescein powder was intratracheally administered in healthy BALB/c mice under anesthesia. Four mice at each time point were sacrificed and the lungs, livers, spleens, and kidneys were excised. The fluorescence images were acquired with an IVIS Spectrum in vivo imaging system with excitation and emission wavelengths of 465 and 540 nm, respectively. The second group received 200 μL of unformulated tamibarotene (0.5 mg/mL dissolved in 0.1% DMSO/PBS) by intraperitoneal (i.p.) administration. Each mouse in both groups received 100 μg of tamibarotene. At specific time point post-administration, five mice in each group were euthanatized by i.p. injection of pentobarbital (90 mg/kg). The blood sample was collected, and the lung tissues were harvested. Tamibarotene in plasma and lung homogenate was extracted by solid phase extraction cartridge (SOLA SAX, Thermo Scientific, USA) and the eluent was dried under a mild stream of nitrogen. The dry residue was reconstituted by HPLC mobile phase (5% acetic acid/acetonitrile 20/80), centrifuged, and the supernatant was assayed by HPLC. The concentration of tamibarotene was quantified against a standard curve ranged from 0.157 to 25 μg/mL with blank plasma or lung homogenates as background. Pharmacokinetic parameters of both groups were analyzed using non-compartmental analysis (NCA) model with Phoenix WinNonLin 7.0 software.

Results

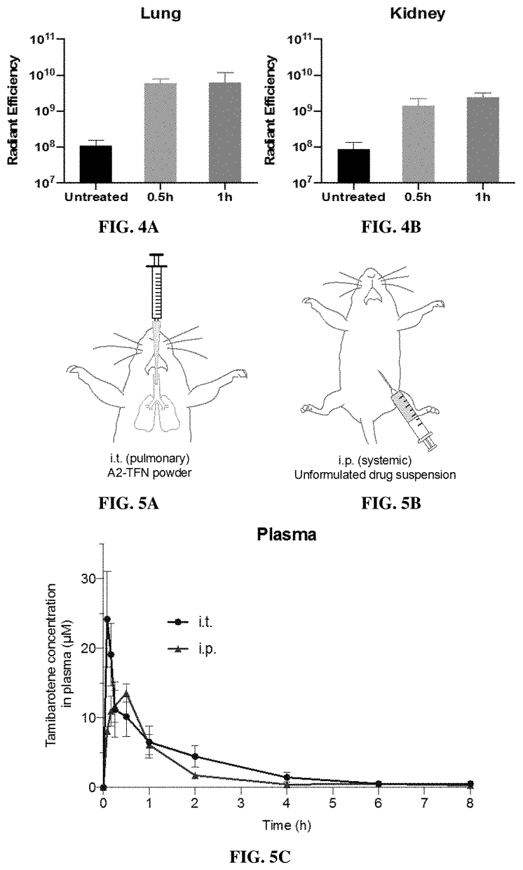

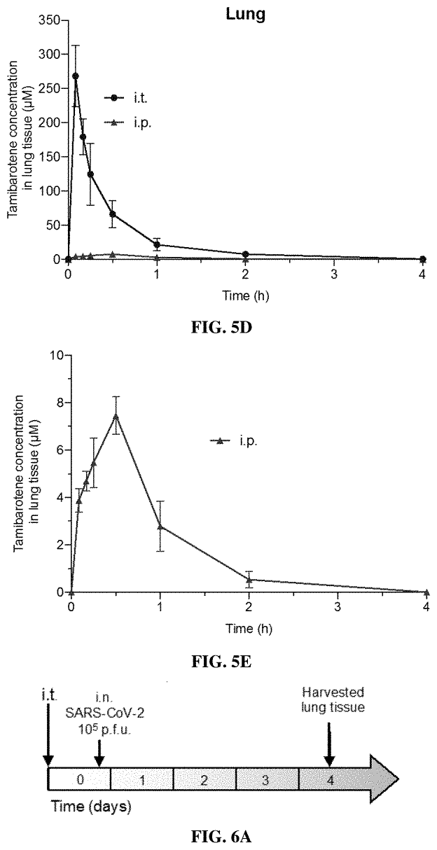

The in vivo fluorescence imaging results show A2-TFN-fluorescein powders in the lung and kidney of the mice at 0.5 and 1 hour after intratracheal administration, demonstrating powder aerosolization after i.t. insufflation (FIGS. 4A and 4B). The pharmacokinetic profile of A2-TFN powder formulation delivered by intratracheal (i.t.) insufflation has been compared with unformulated tamibarotene suspension administered by intraperitoneal (i.p.) injection in healthy BALB/c mice (FIGS. 5A-5E). The pharmacokinetic parameters of i.t. and i.p. groups are shown in Table 2. For the i.t. group, tamibarotene concentration has reached the maximum level in 5 min (Tₘₐₓ=5 min) in both plasma and lung tissues. This observation is in line with the rapid dissolution of A2-TFN powder and hence fast absorption of drug following lung deposition. The drug concentration in plasma and lung tissues has declined with an elimination half-life of 1.7 h and 0.5 h, respectively. While tamibarotene in plasma has maintained above the detectable level up to 8 h, it has become undetectable in lung tissues after 4 h post-administration. For the i.p. group, an absorption phase has been seen in the first 30 min after administration. The Tₘₐₓ for both plasma and lung tissues is 30 min. Cₘₐₓ in plasma following i.p. injection (5.0±0.3 μg/mL, equivalent to ˜14 μM) is lower than that of i.t. administration (8.8±1.7 μg/mL, equivalent to ˜25 μM). This may be due to the slower rate of drug absorption after i.p. injection than i.t. insufflation. The Cₘₐₓ in the lung tissues following i.t. administration is 36-fold higher than that following i.p. administration (p<0.001, Student's t-test). After i.t. administration, the Cₘₐₓ of tamibarotene in the lung is 46-fold higher (for SARS-CoV-2), 838-fold higher (for MERS-CoV), and 159-fold higher (for H1N1) than its antiviral EC₅₀, respectively (Table 2), which demonstrates a favorably high local concentration in the lung for efficient suppression of virus replication. The AUC_{0-8 h} in the lung tissue following i.t. administration is significantly higher (15-fold) than that of i.p. injection (p<0.001, Student's t-test). The AUC_{0-8 h} in plasma following i.t. administration is also higher than that of i.p. injection (p<0.01, Student's t-test).

The pharmacokinetic study shows that pulmonary delivery of A2-TFN formulation exhibits a rapid drug absorption which is superior to the i.p. administration of unformulated drug suspension. Higher bioavailability has been achieved by pulmonary delivery of A2-TFN dry powder while systemic administration of tamibarotene has shown poor lung distribution. The results demonstrate that pulmonary delivery of A2-TFN powder can deliver tamibarotene for local antiviral action in the respiratory tract.

TABLE 2
Pharmacokinetic parameters of tamibarotene following intratracheal (i.t.) administration of A2-
TFN powder formulation and intraperitoneal (i.p.) injection of drug suspension to BALB/c mice.

| Route and | | | | | | | | EC₅₀ (μM) | | |
|---|---|---|---|---|---|---|---|---|---|---|
| dosage | | | Tₘₐₓ | T_{1/2} | AUC_{0-8 h} | AUC_{0-∞}_{_{—}}_{D} | MRT_{0-8 h} | SARS- | MERS- | |
| form | Tissue | Cₘₐₓ | (min) | (h) | (μg · h/mL) | (μg · h/mL) | (h) | CoV-2 | CoV | H1N1 |
| i.t. | Plasma | 25.1 ± 4.8 μM | 5 | 1.7 ± 0.2 | 8.5 ± 1.0* | 1.8 ± 0.2 | 1.8 ± 0.2 | 5.8 ± 1.2 | 0.32 ± 0.026 | 1.68 ± 0.98 |
| (pulmonary) | | (8.8 ± 1.7 μg/mL) | | | | | | | | |
| A2-TFN | Lung | 268.4 ± 44.7 μM | 5 | 0.5 ± 0.1 | 36.0 ± 4.8^{#} | 7.2 ± 1.0 | 0.5 ± 0.1 | | | |
| powder | | (94.2 ± 15.7 μg/mL) | | | | | | | | |
| i.p. | Plasma | 14.2 ± 0.9 μM | 30 | 1.8 ± 0.4 | 6.0 ± 0.5* | 1.5 ± 0.2 | 1.5 ± 0.2 | | | |
| (systemic) | | (5.0 ± 0.3 μg/mL) | | | | | | | | |
| drug | Lung | 7.4 ± 0.9 μM | 30 | 0.5 ± 0.08 | 2.4 ± 0.3^{#} | 0.5 ± 0.06 | 0.6 ± 0.2 | | | |
| suspension | | (2.6 ± 0.3 μg/mL) | | | | | | | | |

Parameters presented include maximum concentration (Cₘₐₓ), time to reach Cₘₐₓ (Tₘₐₓ), half-life (t_{1/2}), area under the curve (AUC) and mean residence time (MRT). These parameters were obtained by non-compartment analysis. For calculation purpose, drug concentration at 4 h post i.p. administration and beyond was treated as zero for non-compartmental analysis (NCA) modeling with manual fitting. EC₅₀ of tamibarotene against MERS-CoV were obtained from previous study (Yuan, et al., Nature Communications 2019, 10, 120). Data were presented as mean ± standard deviation (n = 5).
*p < 0.01,
^{#}p < 0.001, when comparing same tissue between two groups, Student's t-test.

### Example 3. Inhaled Dry Powder Formulations of Tamibarotene Show In Vivo Antiviral Efficacy Against Coronaviruses and Influenza a Virus

Materials and Methods

Animals and Ethics Approval

Healthy female BALB/c mice aged 7˜9 weeks (body weight ˜20 g) were used to investigate the pharmacokinetic profile and anti-influenza effect of tamibarotene in animals following pulmonary delivery. hDPP4 transgenic C₅₇BL/6 mice and golden Syrian hamster were used to investigate the antiviral effect against MERS-CoV and SARS-CoV-2 of tamibarotene following pulmonary delivery, respectively. The mice and hamsters were obtained from the Centre for Comparative Medicine Research of The University of Hong Kong (HKU) and were housed in a 12 h light/dark cycle with food and water available ad libitum. All the animal experiments were performed with the approval from the Committee on the Use of Live Animals in Teaching and Research (CULATR) at (HKU) and following the standard operating procedures of the Biosafety Level 2 and Level 3 animal facilities.

Against SARS-CoV-2

Golden Syrian hamster were used for studying in vivo prophylactic activity of A2-TFN formulation against SARS-CoV-2. The hamsters were divided into three groups (four hamsters per group). In the first two groups, i.t. administration was performed under anesthesia: (i) 200 μL of remdesivir solution (2.5 mg/mL); (ii) 5 mg of A2-TFN powder. Remdesivir was prepared as 100 mg/mL stock in DMSO and further diluted using 12% sulfobutylether-β-cyclodextrin (SBE-β-CD). The third group of hamsters received 200 μL of PBS via i.t. administration as negative control. The dose of remdesivir or tamibarotene was 5 mg/kg per hamster. Two hours after i.t. administration, hamsters were i.n. inoculated with 20 L of virus suspension containing 10⁵ p.f.u. of SARS-CoV-2 under anesthesia by i.p. injection of ketamine (200 mg/kg) and xylazine (10 mg/kg). All hamsters were euthanized on 4 d.p.i. and the lung tissues were collected for further analysis. Viral load in lung homogenates was measured by qRT-PCR method. The lung tissue histopathology of infected hamster was examined by H&E and immunofluorescence staining as we previously described. This experiment was conducted in one independent experiment (n=4 in each group).

Against MERS-CoV

Human dipeptidyl peptidase (hDPP4) transgenic C57BL/6 mice were used for investigating in vivo prophylactic activity of A2-TFN formulation against MERS-CoV. The mice were divided into three groups (five mice per group) for i.t. administration under anesthesia of: (i) 20 μL of unformulated tamibarotene suspension (1 mg/mL); (ii) 1 mg of A2-TFN powder; and (iii) 20 μL of PBS (negative control). The dose of tamibarotene in the first two groups was 5 mg/kg per mouse. Two hours after i.t. administration, hDPP4 mice were i.n. inoculated with 20 μL of virus suspension containing 100 p.f.u. MERS-CoV under anesthesia. All mice were euthanized on 3 d.p.i. and the lung tissues were collected for further analysis. Viral load in lung tissue homogenates was measured by qRT-PCR method. This experiment was conducted in one independent experiment (n=5 in each group).

Against HINI Virus—Prophylactic Protection

BALB/c mice were used to investigate the in vivo activity of A2-TFN formulation against influenza A H1N1 virus. The mice were evaluated in divided into four groups (nine mice per group). Prior to virus inoculation, i.t. administration was performed under anesthesia in each group: (i) 20 μL of zanamivir solution (5 mg/mL); (ii) 50 μL of unformulated tamibarotene suspension (2 mg/mL); and (iii) 1 mg of A2-TFN powder. The dose of zanamivir or tamibarotene was 5 mg/kg per mouse. In the fourth group of mice, 50 μL of PBS was i.t. administered as negative control. At 2 h post administration, all the mice were i.n. inoculated with 20 μL of virus suspension containing 100 p.f.u. of HIN virus under anesthesia. Four mice in each group were euthanatized randomly on 3 d.p.i. and the lung tissues were harvested for viral load assay by qRT-PCR method. Animal survival, clinical symptoms and body weight were monitored for 14 days or until the humane endpoint (body weight loss more than 20%) was reached. The lung tissue histopathology of infected mice was examined by hematoxylin and eosin (H&E) and immunofluorescence staining. This experiment was conducted in two independent experiments (n=9 in each group).

Against H1N1 Virus—Therapeutic Effect

To evaluate the therapeutic effect of tamibarotene formulation against H1N1 virus, BALB/c mice were i.n. inoculated with 20 μL of virus suspension containing 10 p.f.u. of H1N1 virus under anaesthesia. At 4 h post virus challenge, the first therapeutic dose was initiated and the mice were divided into four groups (11 mice per group) to receive 20 μL of each treatment via i.n. administration: (i) zanamivir solution (2 mg/mL); (ii) unformulated tamibarotene suspension (100 μg/mL); (iii) reconstituted A2-TFN solution (100 μg/mL); and (iv) PBS (as negative control). The dose of tamibarotene was 0.1 mg/kg, and the dose of zanamivir was 2 mg/kg. On day 1 and day 2 post-infection, the therapeutic doses were administered twice a day. On 3 d.p.i., four mice in each group were euthanatized randomly and the lung tissues were harvested for viral load assay by qRT-PCR method. Animal survival, clinical symptoms and body weight were monitored for 14 days or until the humane endpoint (body weight loss more than 20%) was reached. This experiment was conducted in two independent experiments (n=11 in each group).

Plaque Reduction Assay for Determination of Antiviral EC₅₀

Plaque reduction assay was performed to plot the 50% antiviral effective dose (EC₅₀). Vero E6 cells were used for SARS-CoV-2 and MDCK cells for influenza A virus. Briefly, cells were seeded at 4×10⁵ cells/well in 12-well tissue culture plates on the day before carrying out the assay. After 24 h of incubation, 50 p.f.u. of SARS-CoV-2 or H1N1 virus were added to the cell monolayer with or without the addition of drug compounds and the plates were further incubated for 1 h at 37° C. in 5% CO₂ before removal of unbound viral particles by aspiration of the media and washing once with DMEM. Monolayers were then overlaid with media containing 1% low melting agarose (Cambrex Corporation, New Jersey, USA) in DMEM and appropriate concentrations of individual compound, inverted and incubated as above for another 72 h. The wells were then fixed with 10% formaldehyde (BDH, Merck, Darmstadt, Germany) overnight. After removal of the agarose plugs, the monolayers were stained with 0.7% crystal violet (BDH, Merck) and the plaques counted. The percentage of plaque inhibition relative to the control (i.e., without the addition of compound) wells were determined for each drug compound concentration. The EC₅₀ was calculated using Sigma plot (SPSS) in an Excel add-in ED50V10. The plaque reduction assay experiments were performed in triplicate and repeated twice for confirmation.

Toxicity Study

HPBCD only powder was prepared according to the same protocol as A2-TFN but without the addition of tamibarotene and tert-butyl alcohol. One milligram of A2-TFN powder or HPBCD powder, or 50 μL of phosphate buffer saline (PBS) was intratracheally administered in healthy BALB/c mice under anesthesia.

Statistical Analysis

Statistical analyses were conducted using GraphPad Prism 8.0 for Student's t-test, ANOVA, and Log-rank (Mantel-Cox) test as indicated in the text or figure captions. The sample size (n) was indicated in the text or figure captions for each experiment. P-value<0.05 was considered as statistically significant throughout this study.

Results

With the aim to develop an inhalation therapy of a safe and potent broad-spectrum antiviral in response to the emerging and re-emerging epidemic and pandemic threats caused by respiratory viral infections, the in vivo prophylactic protection against coronaviruses and influenza A virus has been investigated in animal models following i.t. administration of A2-TFN powder.

Against SARS-CoV-2

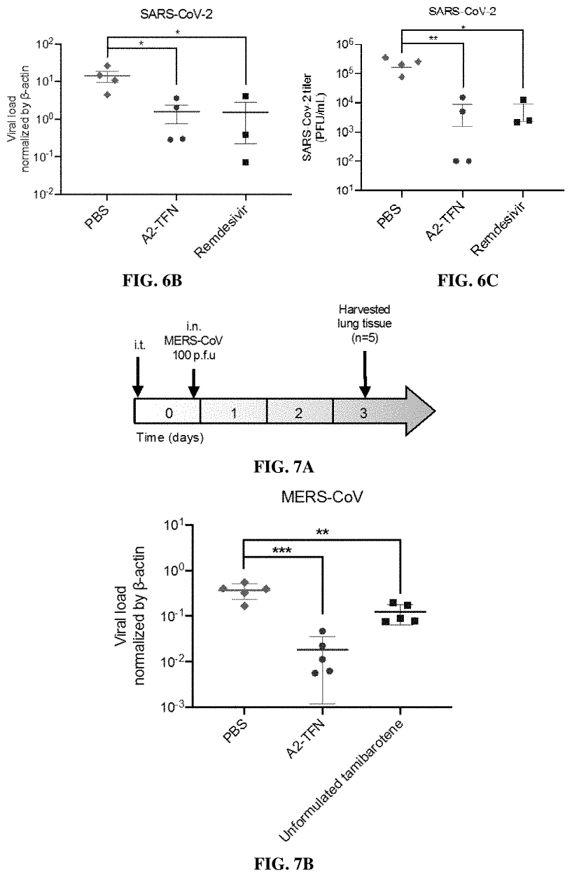

The in vivo antiviral effect of A2-TFN powder against SARS-CoV-2 has been investigated using an established disease model in golden Syrian hamster (FIG. 6A). A single dose of A2-TFN powder or remdesivir solution has been delivered via i.t. administration to the first two groups of hamsters, while the third group has been treated with i.t. administration of phosphate buffer saline (PBS) as vehicle control. The dose of tamibarotene and remdesivir was 5 mg/kg. At 2 h post-administration, when the residual tamibarotene concentration in the lung was estimated to be above the EC₅₀ according to pharmacokinetic study, all the hamsters were intranasally (i.n.) challenged with 10⁵ plaque-forming units (p.f.u.) SARS-CoV-2 (FIG. 6A). The vehicle-treated control hamsters have developed the clinical signs of lethargy, hunched back posture, and rapid breathing starting from 2 d.p.i., whereas the hamsters treated with A2-TFN or remdesivir have not developed clinical symptoms. The highest viral load and the most prominent histopathological change are expected on day 4 post-infection in this model. The lung tissues of hamsters have been harvested to examine whether A2-TFN powder and remdesivir can protect the animal from SARS-CoV-2 infection. As shown in FIGS. 6B and 6C, the viral RNA load and viral titer in the lung tissues of hamster receiving i.t. administration of A2-TFN and remdesivir is significantly lower than that of PBS-treated hamster (p<0.05, one-way ANOVA with post-hoc multiple comparison). There is no difference in terms of both the viral load and viral titer in the hamster lung tissue between A2-TFN powder and remdesivir treatment (p>0.05, one-way ANOVA). Histopathological study of the lung tissues at 4 d.p.i. has been performed to examine the disease severity (data not shown). Diffuse lung damage, alveolar collapse and massive inflammatory cell infiltration and exudation have been observed in the lung tissue of hamsters treated with PBS. In contrast, the lung tissues of hamster treated with A2-TFN powder and remdesivir show ameliorated histopathological morphology with a mild degree of bronchiolar and alveolar cell infiltration. Immunofluorescence staining results show that although SARS-CoV-2 nucleocapsid protein (NP) expression is seen in focal bronchiolar epithelial cells of both A2-TFN powder- and remdesivir-treated hamsters, the expression of NP in alveolar region has been considerably reduced in these two groups when compared with PBS control group, showing a restriction of SARS-CoV-2 spread upon drug treatment (data not shown). These results show that pulmonary delivery of A2-TFN powder in hamster can provide antiviral protection against SARS-CoV-2 and improve viral infection-associated symptoms, with a protective effect comparable to that of remdesivir, an FDA-approved antiviral for the treatment of COVID-19.

The pan-coronavirus antiviral potential of the inhaled tamibarotene powder in human dipeptidyl peptidase 4 (hDPP4) transgenic C₅₇BL/6 mice model has been investigated. The results show that a single dose of A2-TFN powder given by i.t. insufflation prior to virus challenge can confer some protection to the mice from MERS-CoV infection, as evident by the significantly decreased viral load in the lung (FIGS. 7A and 7B).

Against HIN Virus

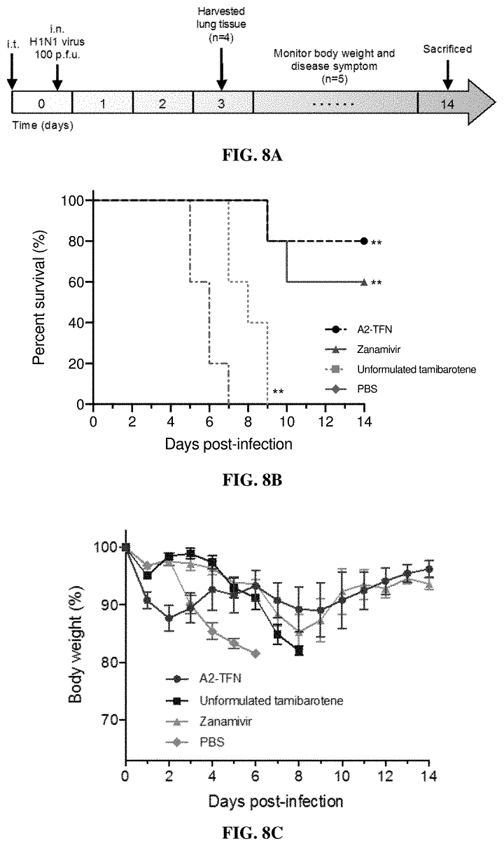

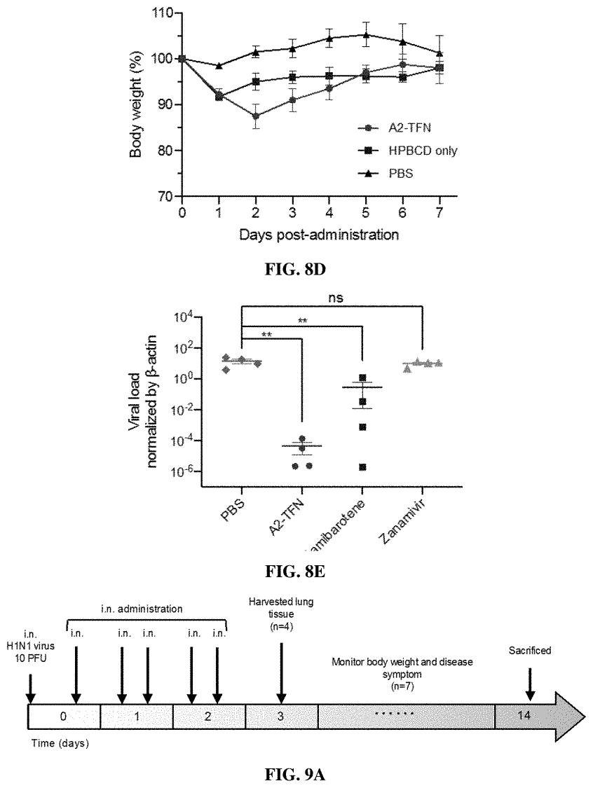

The prophylactic anti-influenza activity of tamibarotene formulation in BALB/c model has been evaluated (FIG. 8A). Prior to virus inoculation, a single dose of A2-TFN powder, unformulated tamibarotene suspension, zanamivir solution or PBS has been delivered to the lung of mice via i.t. administration. The dose of tamibarotene and zanamivir was 5 mg/kg. At 2 h post-administration, the animals were inoculated i.n. with 100 p.f.u. H1N1 virus and monitored for 14 days (FIG. 8A). As shown in FIG. 8B, at 14 days post-infection (d.p.i.), the survival rates of A2-TFN group and zanamivir group are 80% and 60%, respectively, while all the mice in PBS group and unformulated tamibarotene group have reached humane endpoint before 7 and 9 d.p.i., respectively. The survival rate of A2-TFN group, zanamivir group and unformulated tamibarotene group is significantly higher than that of PBS group (p<0.01, log-rank test). From 1 to 3 d.p.i., a decrease in body weight (˜10%) has been observed in mice treated with A2-TFN powder (FIG. 8C), which is considered to be a side effect of powder insufflation to the lung of mice. Given the fact that this powder formulation has an aerodynamic size suitable for effective lung deposition in human, the side effect after i.t. administration in mice is expected due to the considerable anatomical difference between human and rodent. Starting from 4 d.p.i., the body weight of mice treated with A2-TFN powder has risen back to around 95% of baseline level, showing the recovery of animals.

The body weight of uninfected mice has been monitored following intratracheal administration of spray freeze dried HPβCD and A2-TFN powder (FIG. 8D). A similar level of weight loss has been observed after day 1 post-administration for both groups, probably because the aerosol particles were designed for humans instead of mice. On 2 d.p.i., the body weight of A2-TFN-treated mice have continued to decrease while HPBCD-treated mice have started recovering. From 3 d.p.i, A2-TFN treated mice have gradually recovered and eventually returned to comparable level as HPBCD-treated mice. The HPβCD group have recovered more rapidly than A2-TFN group. Consistent with the body weight trend, although inflammatory cell infiltration was observed in the lung tissue of both groups on 3 d.p.i., improved histopathological morphology was seen in lung tissue on 7 d.p.i., demonstrating the recovery of the animals (data not shown). No marked toxicity has been observed in the lung histopathology of the uninfected mice (data not shown). As shown in FIG. 8E, on day 3 post-challenge, the viral RNA load in lung tissues harvested from mice treated with A2-TFN powder and unformulated tamibarotene are significantly lower than that of PBS-treated mice (p<0.01, one-way ANOVA). There is no significant difference in viral load between PBS group and zanamivir group.

Histopathological examination of hematoxylin and eosin (H&E)-stained lung tissues has been conducted at 3 d.p.i. (data not shown). Severe alveolar damage and interstitial inflammatory infiltration have been observed in the lung tissue of mice treated with PBS and unformulated tamibarotene, albeit to a less extent for the latter. In contrast, the alveolar damage and interstitial infiltration have been alleviated in the lung tissues of mice treated with A2-TFN powder and zanamivir. Immunofluorescence staining demonstrates that massive viral PA protein expression has been seen in the diffuse alveolar areas and in the focal bronchiolar epithelial cells from the mice treated with PBS (data not shown). In the lung tissues of mice treated with unformulated tamibarotene and zanamivir, viral PA protein expression in diffuse alveolar region has been diminished but remained noticeable in bronchiolar epithelial cells. The expression of PA protein has been largely suppressed in both alveolar and bronchiolar region in the lung tissue of A2-TFN-treated mice. These results demonstrate that a single prophylactic dose of A2-TFN via i.t. administration can protect the mice from the highly pathogenic H1N1 virus challenge with an efficacy comparable to or better than the commercially available anti-influenza drug zanamivir.

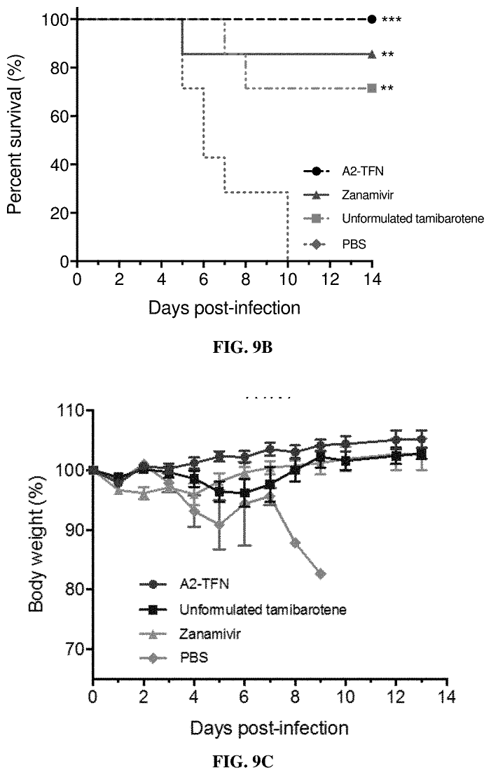

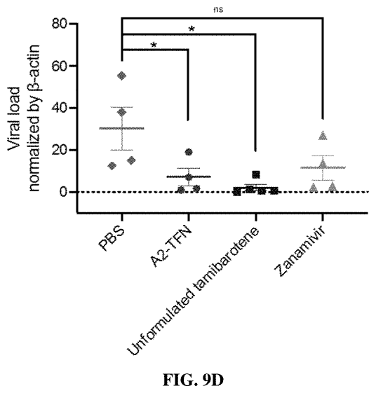

With the excellent protection provided by a single prophylactic dose of inhaled tamibarotene powder against H1N1 virus, the therapeutic effect of tamibarotene powder reconstitution administered by i.n. administration has been investigated (FIG. 9A). After i.n. inoculation with 10 p.f.u. of H1N1 virus, the mice received reconstituted A2-TFN solution, unformulated tamibarotene suspension, or zanamivir solution via i.n. administration. The dose of tamibarotene and zanamivir was 0.1 mg/kg and 2 mg/kg, respectively. The fourth group of mice received 20 μL of PBS solution by i.n. administration as negative control. A total of five therapeutic doses have been delivered. As shown in FIG. 9B, at 14 d.p.i., the survival rate of A2-TFN reconstitution group has reached 100%, whereas survival rates of zanamivir group and unformulated tamibarotene group are 86% and 71%, respectively. With all the mice in PBS group reaching humane endpoint by 10 d.p.i., the survival rate of mice in A2-TFN group, zanamivir group and unformulated tamibarotene group is significantly higher than that of PBS group (p<0.01, Log-rank test). No remarkable body weight loss and disease symptom have been recorded from the mice treated with A2-TFN reconstitution throughout the 14-day monitoring (FIG. 9C). On 3 d.p.i., the lung tissues of mice in each group were harvested for viral RNA load assay (FIG. 9D). The viral load in A2-TFN reconstitution group and unformulated tamibarotene group has been significantly lower than that of PBS group (p<0.05, one-way ANOVA), while the viral load in zanamivir group and PBS group have not been significantly different (p>0.05, one-way ANOVA). The above results show that, in addition to single dose prophylaxis of tamibarotene powder delivered via i.t. route, i.n. administration of reconstituted tamibarotene powder solution and unformulated tamibarotene suspension can also protect the mice from H1N1 viral infection.

In summary, the broad-spectrum protective antiviral efficacy of A2-TFN powder formulation against SARS-CoV-2, MERS-CoV and H1N1 virus following single prophylactic dose administrated via the pulmonary route in vivo, as well as therapeutic effect against H1N1 via the intranasal route, have been demonstrated. The efficacy of SFD tamibarotene formulation and its reconstitution are superior to the unformulated tamibarotene and comparable to or better than the commercially available antiviral agents remdesivir (against SARS-CoV-2) and zanamivir (against H1N1 virus).

### REFERENCES

Cheng, et al., Clin Microbiol Rev 2012, 25, 223.
Cheng, et al., Clin Microbiol Rev 2007, 20, 660.
Chan, et al., Clin Microbiol Rev 2015, 28, 465.
Chan, et al., Lancet 2020, 395, 514.
Peiris, et al., Nat Med 2004, 10, S88.
Zumla, et al., Lancet 2015, 386, 995.
Wang, et al., 3rd, AAPS PharmSciTech 2020, 21, 225.
Zhao, et al., Front Immunol 2020, 11, 602256.
T.R.C. Group, New England Journal of Medicine 2020, 384, 693.
Weinreich, et al., N Engl J Med 2021, 384, 238.
Pan, et al., N Engl J Med 2021, 384, 497.
Altan-Bonnet, Trends Cell Biol 2017, 27, 201.
Munger, et al., Nat Biotechnol 2008, 26, 1179.
Yuan, et al., Nature Communications 2019, 10, 120.
Heaton, et al., PNAS 2010, 107, 17345.
Mayer, et al., Front Immunol 2019, 10, 1533.
Kleinfelter, et al., mBio 2015, 6, e00801.
Petersen, et al., PLoS Pathog 2014, 10, e1003911.
Yuan, et al., Viruses 2020, 12, 628.
Miwako, et al., Drugs of today (Barcelona, Spain: 1998) 2007, 43, 563.
Zhou, et al., Adv Drug Deliv Rev 2015, 85, 83.
Reychler, et al., Respir Med Res 2020, 78, 100778.
Davis, et al., Nat Rev Drug Discov 2004, 3, 1023.
Vishali, et al., J Control Release 2019, 300, 93.
Liang, et al., International journal ofpharmaceutics 2018, 552, 67.
Liao, et al., Int J Pharm 2020, 584, 119444.
Wanning, et al., Int J Pharm 2015, 488, 136.
Chan, Journal of aerosol medicine 2006, 19, 21.
Chaurasiya and Zhao, Pharmaceutics 2021, 13, 31.
Hoppentocht, et al., Advanced Drug Delivery Reviews 2014, 75, 18.
Levy, et al., Adv Ther 2019, 36, 2547.
Rogliani, et al., Respir Med 2017, 124, 6.
Esclusa-Diaz, et al., International Journal of Pharmaceutics 1996, 143, 203.
Rodier, et al., European journal of pharmaceutical sciences 2005, 26, 184.
Schittny, et al., Drug Deliv 2020, 27, 110.
Chan, et al., Clin Infect Dis 2020, 71, 2428.
Yuan, et al., Nat Microbiol 2020, 5, 1439.
Ghaebi, et al., J Cell Physiol 2020, 235, 9098.
Haynes, et al., Sci Transl Med 2020, 12, eabe0948.
Colombo, et al., in Inhalation Drug Delivery, 2013, 169.
de Boer, et al., Expert Opinion on Drug Delivery 2017, 14, 499.
Andrade, et al., Adv Drug Deliv Rev 2013, 65, 1816.
Chennakesavulu, et al., Asian J Pharm Sci 2018, 13, 91.
Gaspar, et al., Int J Pharm 2019, 556, 117.
Nemati, et al., AAPS PharmSciTech 2019, 20, 120.
Parumasivam, et al., Eur J Pharm Sci 2016, 88, 1.
Thomas, et al., Mol Pharm 2011, 8, 405.
Beck-Broichsitter, et al., J Control Release 2012, 161, 214.
Pilcer and Amighi, Int J Pharm 2010, 392, 1.
Dufour, et al., Int J Pharm 2015, 495, 869.
Evrard, et al., J Control Release 2004, 96, 403.
Guan, et al., Molecules 2020, 25.
Loftsson, et al., Expert Opinion on Drug Delivery 2005, 2, 335.
Matilainen, et al., J Control Release 2008, 126, 10.
Zhang, et al., Clinical Infectious Diseases 2020.
Marques, et al., Dissolution Technol 2011, 18, 15.
Yuan, et al., Sci Adv 2020, 6, eaba7910.
Chan, et al., Clin Infect Dis 2020, 71, 2139.
Qiu, et al., J Vis Exp 2020, 161, e61469.
Liao, et al., International journal ofpharmaceutics 2019, 560, 144.
Yuan, et al., Pharmacol Res 2020, 159, 104960.
Yuan, et al., J Antimicrob Chemother 2016, 71, 2489.
Yuan, et al., Antiviral Res 2017, 137, 58.
Riva, et al., Nature 2020, 586, 113.

Unless defined otherwise, all technical and scientific terms used herein have the same meanings as commonly understood by one of skill in the art to which the disclosed invention belongs.

Those skilled in the art will recognize, or be able to ascertain using no more than routine experimentation, many equivalents to the specific embodiments of the invention described herein. Such equivalents are intended to be encompassed by the following claims.

We claim:

1. A dry powder formulation for inhalation or intratracheal administration and/or for intranasal administration comprising particles comprising
a retinoid or a retinoid derivative, or a combination thereof; and
a β-cyclodextrin or a β-cyclodextrin derivative, or a combination thereof,
wherein the amount of the β-cyclodextrin or the β-cyclodextrin derivative, or the total amount of the β-cyclodextrin and β-cyclodextrin derivative is at least 20 wt % of the total amount of the retinoid and/or retinoid derivative and the β-cyclodextrin and/or β-cyclodextrin derivative.

2. The dry powder formulation of claim 1, wherein the particles are porous and spherical in shape.

3. The dry powder formulation of claim 1, wherein the retinoid and/or retinoid derivative form a complex with the β-cyclodextrin and/or β-cyclodextrin derivative via hydrophobic interactions.

4. The dry powder formulation of claim 1, wherein the retinoid and/or retinoid derivative are/is in an amorphous form.

5. The dry powder formulation of claim 1, wherein the particles comprise retinol, tretinoin, isotretinoin, alitretinoin, etretinate, acitretin, adapalene, bexarotene, tazarotene, or tamibarotene, or a combination thereof.

6. The dry powder formulation of claim 1, wherein the particles comprise a β-cyclodextrin derivative, and wherein the β-cyclodextrin derivative is 2-hydroxypropyl-β-cyclodextrin, methyl-β-cyclodextrin, or sulfobutylether β-cyclodextrin, or a combination thereof.

7. The dry powder formulation of claim 1, wherein particles comprise tamibarotene and 2-hydroxypropyl-β-cyclodextrin.

8. The dry powder formulation of claim 1 further comprising a pharmaceutically acceptable excipient, an additional active agent, or a combination thereof.

9. The dry powder formulation of claim 8, wherein the pharmaceutically acceptable excipient is an amino acid, a peptide, a lipid, a protein, a chelating agent, a salt, a taste masking agent, a cation, or a polymer, or a combination thereof.

10. The dry powder formulation of claim 8, wherein the amount of the pharmaceutically acceptable excipient is in a range from 0.1 wt % to 20 wt %, from, from 0.1 wt % to 15 wt %, from 1 wt % to 12 wt %, from 1 wt % to 10 wt %, from 1 wt % and 15 wt %, from 2 wt % to 20 wt %, from 2 wt % to 15 wt %, from 2 wt % to 10 wt %, from 3 wt % to 20 wt %, from 3 wt % to 15 wt %, or from 3 wt % to 10 wt % of the dry powder formulation.

11. The dry powder formulation of claim 8, wherein the additional active agent is an anti-viral agent or anti-inflammatory agent, or a combination thereof.

12. The dry powder formulation of claim 1, wherein the dry powder formulation is for inhalation, and wherein the particles have a mass median aerodynamic diameter (“MMAD”) of <5 μm, <4 μm, <3.5 μm, <3 μm, <2.5 μm, or <2 μm.

13. The dry powder formulation of claim 12, wherein the particles have a volumetric mean diameter that is larger than the MMAD of the particles, and wherein the volumetric mean diameter of the particles is >4 μm, >5 μm, >8 μm, >10 μm, >12 μm, >15 μm, in a range from 4 μm to 20 μm, from 4 μm to 15 μm, or from 4 μm to 15 μm.

14. The dry powder formulation of claim 12, wherein the particles have a fine particle fraction>40%, >45%, >50%, >55%, >60%, or >65% in cascade impactor study.

15. The dry powder formulation of claim 1, wherein the dry powder formulation is for intranasal administration, and wherein the particles have a mass median aerodynamic diameter>9 μm, >9.5 μm, >10 μm, or >10.5 μm.

16. The dry powder formulation of claim 15, wherein the particles have a volumetric mean diameter of >50 μm, >55 μm, >60 μm, >65 μm, in a range from 50 μm to 80 μm, from 50 μm to 75 μm, or from 50 μm to 70 μm.

17. The dry powder formulation of claim 15, wherein the particles have a fraction of particles>9 μm of >40%, >45%, >50%, >55%, or >60% in Andersen Cascade Impactor (“ACI”) study.

18. A delivery system comprising an inhaler and the dry powder formulation of claim 1 in a unit dosage form.

19. The delivery system of claim 18, wherein the inhaler is a dry powder inhaler or a pressurized metered dose inhaler.

20. The delivery system of claim 18, wherein the total amount of the retinoid and/or retinoid derivative in the unit dosage form is in a range from about 0.1 mg to about 50 mg, from about 0.5 to about 50 mg, or from about 1 to about 50 mg.

21. The delivery system of claim 18, wherein the emitted fraction of the particles is >65%, >70%, >75%, >80%, >85%, >90%, >92%, or >95%.

22. A delivery system comprising a nasal device and a solution or suspension formed by the dry powder formulation of claim 1 in a unit dosage form and a solvent, wherein the particles of the dry powder formulation are dissolved or suspended in the solvent.

23. The delivery system of claim 22, wherein the nasal device is a metered dose spray pump, an atomiser, a syringe, a bulb, a canister, a pressurized container, a spray can, or a nebulizer.

24. The delivery system of claim 22, wherein the total amount of the retinoid and/or retinoid derivative in the unit dosage form is in a range from about 0.1 mg to about 50 mg, from about 0.5 to about 50 mg, or from about 1 to about 50 mg.

25. The delivery system of claim 22, wherein the emitted fraction of the particles is >85%, >90%, or >95%.

26. A method of making the dry powder formulation of claim 1 comprising
(i) mixing a retinoid and/or a retinoid derivative and a β-cyclodextrin and/or a β-cyclodextrin derivative, and optionally a pharmaceutically acceptable excipient and/or an additional active agent, in a solvent to form a liquid feed; and
(ii) spray-drying or spray-freeze drying the liquid feed to form particles containing the retinoid and/or a retinoid derivative and the β-cyclodextrin and/or β-cyclodextrin derivative, and optionally the pharmaceutically acceptable excipient and/or the additional active agent.

27. The method of claim 26, wherein following step (ii), the production yield of the particles is at least 40 wt %, at least 50 wt %, at least 60 wt %, at least 65 wt %, at least 70 wt %, at least 75 wt %, at least 80 wt %, at least 85 wt %, at least 90 wt %, in a range from about 40 wt % to about 95 wt %, from about 55 wt % to about 95 wt %, from about 55 wt % to about 95 wt %, from about 60 wt % to about 95 wt %, or from about 65 wt % to about 95 wt %.

28. The method of claim 26, wherein during step (ii), the liquid feed is sprayed through a two-fluid nozzle.

29. A method of preventing, treating, or ameliorating symptom(s) associated with a respiratory viral infection in a subject, the method comprising (i) administering to the subject the dry powder formulation of claim 1.

30. The method of claim 29, wherein the subject is a mammal having or at the risk of having sever acute respiratory syndrome, Middle East respiratory syndrome, Coronavirus Disease, or a flu caused by an influenza virus, or a combination thereof.

31. The method of claim 29, wherein in step (i), the dry powder formulation is administered by inhalation or intratracheal administration.

32. The method of claim 29, wherein prior to step (i), the dry powder formulation is mixed with a solvent to form a solution or suspension, and wherein in step (i), the solution or suspension is administered by intranasal administration.

33. The method of claim 29 further comprising repeating step (i) every hour, every 2 hours, every 5 hours, every 8 hours, every day, every 2 days, every 3 days, every 5 days, every 7 days, every 10 days, every two weeks, or every month.

34. The method of claim 33, wherein step (i) is repeated for a period between one day and 6 months, between one day and 3 months, between one and thirty days, between one and ten days, between one and three days, or during one day.

35. The method of claim 29, wherein following a single administration step or all of the administration steps, an effective amount of the retinoid and/or retinoid derivative is delivered to the lower and/or upper respiratory tract of the subject.

36. The method of claim 35, wherein the subject is infected by a severe acute respiratory syndrome coronavirus, a Middle East respiratory syndrome coronavirus, and/or an influenza virus after the single administration step or all of the administration steps, and wherein the effective amount of retinoid and/or retinoid derivative is effective to reduce the viral load in the lung of the subject compared to the viral load in the lung of a control, to reduce the degree of lung damage compared to the degree of lung damage in the control, and/or to reduce the expression level of a viral protein compared to the expression of the viral protein in the control.

37. The method of claim 35, wherein the subject is infected by a severe acute respiratory syndrome coronavirus, a Middle East respiratory syndrome coronavirus, and/or an influenza virus prior to the single administration step or all of the administration steps, and wherein the effective amount of retinoid and/or retinoid derivative is effective to increase the survival rate and/or to reduce the viral load in the lung of the subject, compared to the survival rate and/or viral load in the lung of a control.

38. The method of claim 29, wherein during step (i), the dosage of the retinoid and/or retinoid derivative in the dry powder formulation or the solution or suspension formed from the dry powder formulation administered is in a range from about 0.1 mg to about 100 mg, from about 0.1 mg to about 50 mg, from about 0.1 mg to about 10 mg, from about 0.1 mg to about 5 mg, from about 0.5 mg to about 100 mg, from about 0.5 μg to about 50 mg, from about 0.5 mg to about 20 mg, from about 0.5 mg to about 10 mg, from about 1 mg to about 100 mg, from about 1 mg to about 50 mg, from about 1 μg to about 20 mg, from about 1 mg to about 10 mg, from about 5 mg to about 100 mg, or from about 5 mg to about 50 mg per kg of the subject.
